(19) United States
(12) Patent Application Publication
(10) Pub. No.: US 2021/0379062 A1
(43) Pub. Date: Dec. 9, 2021

(54) The use of a H3R inverse agonist for the treatment of excessive daytime sleepiness associated with parkinson's disease (PD)

(71) Applicant: Novartis AG, Basel (CH)

(72) Inventors: Donald Johns, Woburn, MA (US); Judit Sovago, Basel (CH)

(21) Appl. No.: 17/284,011

(22) PCT Filed: Oct. 10, 2019
(86) PCT No.: PCT/IB2019/058651

§ 371 Date: Apr. 9, 2021

Related U.S. Application Data
(60) Provisional application No. 62/744,153, filed on Oct. 11, 2018.

(51) Int. Cl. A61K 31/501; A61K 31/198; A61K 31/4745; A61K 31/4045; A61K 31/277; A61K 31/165; A61K 31/426; A61P 25/26

(52) U.S. Cl. CPC A61K 31/501; A61K 31/198; A61K 31/4745; A61P 25/26; A61K 31/277; A61K 31/165; A61K 31/426; A61K 31/4045

(57) ABSTRACT

The invention relates to the use of Compound (I), as defined herein, or pharmaceutically acceptable salt thereof, in the treatment of excessive daytime sleepiness associated with Parkinson's disease.

### FIELD OF THE INVENTION

The invention relates to the use of Compound (I), as defined herein, or pharmaceutically acceptable salt thereof, in the treatment of excessive daytime sleepiness associated with Parkinson's disease.

### BACKGROUND OF THE INVENTION

Parkinson's disease (PD) is a slowly progressive neurodegenerative disorder, which is caused by the degeneration of dopaminergic neurons in the substantia nigra and which affects about 1% of the population over 60 years of age (Rodrigues, T. M. et al., Parkinson and related disorders, 2016, 27: 25-34). Motor symptoms associated with PD include muscle rigidity, akinesia and dystonia. Patients with Parkinson's disease frequently have, in addition, non-motor symptoms such as sleep disorders [e.g. excessive daytime sleepiness (EDS)], cognitive function impairment [e.g. such as deficits in attention, executive function, learning and visuospatial], fatigue, olfactory dysfunction and autonomic dysfunction (e.g. nocturia). Moreover, it is common that Parkinson's disease patients present comorbidity with psychiatric disorders, such as depression, anxiety and psychosis.

Sleep disorders associated with PD include: a) nocturnal manifestations [e.g. insomnia, parasomnias, such as parasomnias associated or not with rapid eye movement (REM), sleep-related breathing disorders, and sleep-related movement disorders, such as restless leg syndrome (RLS) and periodic limb movement disorder (PLMD)] and b) diurnal manifestations [e.g. excessive daytime sleepiness (EDS) and sudden sleep attacks]. They affect up to 90% of PD patients, have a detrimental effect on their quality of life (Aarsland, D. et al., Adv. Neurol. 2005, 96, 56-64) and, furthermore, pose significant safety risks (e.g. increased risk of sleepiness-related accidents). The etiology is multifactorial and it mainly involves the degeneration of the sleep-regulating structures (Int. Rev. Neurobiol., 2017; 133: 719-742).

Excessive daytime sleepiness (EDS) is a common symptom in PD patients, with prevalence ranging from 15 to 50% (Suzuki, K., et al., Parkinson's disease, Vol. 2011, Article ID 219056). Importantly, EDS can occur in the early stages of PD and its incidence increases with disease progression. Due to the multifactorial nature of EDS, the causal mechanism is far from being stablished. However, it has been suggested that different factors may cause EDS in PD patients: it may be primary to the disease's progression itself (i.e. due to the neurodegenerative process of PD itself dysregulating the circadian sleep-wake rhythm) and secondary to nocturnal sleep disruption from coexistent sleep disorders (e.g. PLM) or due to the use of pharmacological therapy, such as antidepressants (e.g. serotonin reuptake inhibitors, serotonin-norepinephrine reuptake inhibitors or β-blockers), antihistamines, antipsychotics or sedatives, in particular dopamine agonists (Dhawan, V. et. al., Age and Aging, 2006, 35:220-228). Furthermore, dopamine replacement therapy, which is the most common treatment for Parkinson's disease (e.g. at early stages of the disease), is known to be associated with increasing excessive daytime sleepiness. Accordingly, this disabling condition is probably caused by a combination of the neurodegenerative process affecting most ascending arousal systems in the brain and the effects of dopaminergic drugs (O'Suilleabhain and Dewey, Arch Neurol, 2002, 59(6), 986-989; Fabbrini et al, Mov Disord, 2002, 17(5), 1026-1030). In contrast, other factors (such as mood disorders or cognitive decline) do not show consistent association with EDS and are therefore unlikely to contribute to the pathogenesis of EDS in PD patients.

Subjective sleepiness is present in about 33.5-54% of Parkinson's disease patients, compared to 16-19% of controls (Chahine et al, Sleep Med Rev, 2017, 33-50). EDS has a negative impact on quality of life as it affects activities of daily living (Visser et al, J Neurol, 2008, 255, 1580-1587; Havlikova et al, J Neurol, 2011, 258(12), 2222-2229) and increases the risk of falls (Spindeler et al, J Parkinsons Dis, 2013, 3(3), 387-391) and automobile accidents (Chaudhuri et al, Drug Saf, 2002, 25(7), 473-483; Meindorfner et al, Mov Disord, 2005, 20(7), 832-842; Uc et al, Neurology, 2006, 67(10), 1774-1780). EDS in PD also has a negative impact on caregiver burden (Ozdilek and Gunai, J. Neuropsychiatry Clin Neurosci, 2012, 24(4), 478-483).

Despite the high prevalence of sleep disorders in PD patients, and their detrimental effect on quality of life, few clinical trials have been conducted. The H3R inverse agonist known as bavisant is currently in clinical trials for the treatment of PD-EDS (ClinicalTrials.gov Identifier: NCT03194217) and the H3R inverse agonist known as pitolisant (also named tiprolisant) completed clinical trials for the treatment of PD-EDS (ClinicalTrials.gov Identifiers: NCT00642928, NCT01066442, NCT01036139; Arnulf I. in European Neuropsychopharmacology. 22nd ECNP Congress, Istanbul Turkey_Conference Publication: 19 SUPPL. 3, p. S204, 2009). At present, there is no approved medication available for the treatment of PD-EDS.

Currently available drugs used to modulate wakefulness and sleep, such as drugs that promote wakefulness, suffer from a number of shortcomings, for example, modafinil, methylphenidate, sodium oxybate and pitolisant have showed variable benefit in clinical studies. Modafinil {i.e. 2-(benzhydrylsulfinyl)acetamide, or 2-[(diphenylmethyl)sulfinyl]acetamide} is a wake-promoting agent, whose structure is disclosed in U.S. Pat. No. 4,177,290, and which has been approved by the US Food and Drug Administration (FDA) for use in the treatment of narcolepsy and shift work disorder. However, in Europe, the European Medicines Agency (EMA) recommended the restriction of all modafinil's indications except for narcolepsy due to unfavorable risk/benefit profile; for example, it is associated with increased risk for development of skin or hypersensitivity reactions and neuropsychiatric disorders. In addition, particular cardiovascular risks have also been associated with modafinil (EMA press release dated 22 Jun. 2010: EMA/459173/2010). The use of modafinil for the treatment of excessive sleepiness associated with PD has been disclosed in: (1) Sleep, 2002, 25:905-9 and in (2) J. Neurol. Neurosurg. Psychiatry, 2005, 76:1636-9. A clinical trial showed that modafinil, 100-200 mg/day after two weeks treatment, improved the Epworth Sleeping Scale scores but not sleep latency in the Maintenance of Wakefulness Test [i.e. reference (1) here above], while another trial with modafinil 200-400 mg/day for four weeks showed no improvement of Epworth Sleeping Scale scores [i.e. reference (2) here above]. Accordingly, there is a need to identify new therapeutic agents that can be used to treat EDS associated with PD, in particular drugs that are effective and have a favorable (e.g. more favorable) risk/benefit profile.

### SUMMARY OF THE INVENTION

In one aspect, the invention relates to uses of the H3R inverse agonist named 1-(1-methyl-6-oxo-1,6-dihydropyridazin-3-yl)piperidin-4-yl 4-cyclobutylpiperazine-1-carboxylate, which herein below is also referred to as Compound (I):
The use of 1-(1-methyl-6-oxo-1,6-dihydropyridazin-3-yl)piperidin-4-yl 4-cyclobutylpiperazine-1-carboxylate, or pharmaceutically acceptable salt thereof, in the manufacture of a medicament for a treatment promoting wakefulness in a Parkinson's disease patient;
The use of 1-(1-methyl-6-oxo-1,6-dihydropyridazin-3-yl)piperidin-4-yl 4-cyclobutylpiperazine-1-carboxylate, or pharmaceutically acceptable salt thereof, in the manufacture of a medicament for the treatment of excessive daytime sleepiness associated with Parkinson's disease;
The use of 1-(1-methyl-6-oxo-1,6-dihydropyridazin-3-yl)piperidin-4-yl 4-cyclobutylpiperazine-1-carboxylate, or pharmaceutically acceptable salt thereof, in the manufacture of a medicament for the treatment of excessive daytime sleepiness associated with dopamine replacement therapy in Parkinson's disease;
The use of 1-(1-methyl-6-oxo-1,6-dihydropyridazin-3-yl)piperidin-4-yl 4-cyclobutylpiperazine-1-carboxylate, or pharmaceutically acceptable salt thereof, in the manufacture of a medicament for the treatment of cognitive function impairment associated with Parkinson's disease

In a second aspect, the invention also relates to a combination of the H3R inverse agonist named 1-(1-methyl-6-oxo-1,6-dihydropyridazin-3-yl)piperidin-4-yl 4-cyclobutylpiperazine-1-carboxylate:
A combination comprising 1-(1-methyl-6-oxo-1,6-dihydropyridazin-3-yl)piperidin-4-yl 4-cyclobutylpiperazine-1-carboxylate, or pharmaceutically acceptable salt thereof, and at least one active ingredient selected from the group consisting of levodopa, the combination of levodopa and pergolide, the combination of levodopa and cabergoline, the combination of levodopa and ropinirole, the combination of levodopa and carbidopa, the combination of levodopa and entacapone, the combination of levodopa and benserazide, the combination of levodopa and pramipexole, amantadine, selegiline, rasagiline, entacapone, ramelteon, melatonin, zolpidem, eszopiclone, zopiclone, brotizolam, trazodone, doxepin, darifenacin, solifenacin, tolterodine, pregabalin, gabapentin, enacarbil, paroxetine, donepezil, rivastigmine, desipramine, carbamazepine, clonazepam, lorazepam, triazolam, temazepam, flurazepam, cabergoline, rotigotine, suvorexant, pergolide, pramipexole, cabergoline, ropinirole, carbidopa, benserazide, clozapine, quetiapine, primavanesrin, duloxetine, mirtazapine, nortriptyline, venlafaxine, modafinil, armodafinil, caffeine, methylphenidate, dextroamphetamine and sodium oxybate; or pharmaceutically acceptable salts thereof; in particular modafinil, armodafinil, caffeine, methylphenidate, dextroamphetamine, alprazolam, solriamfetol, and sodium oxybate, or pharmaceutically acceptable salts thereof.

In a further aspect, the invention relates to uses of the above combination:
The use of the above combination, in the manufacture of a medicament for a treatment promoting wakefulness in a Parkinson's disease patient;
The use of the above combination, in the manufacture of a medicament for the treatment of excessive daytime sleepiness associated with Parkinson's disease;
The use of the above combination, in the manufacture of a medicament for the treatment of excessive daytime sleepiness associated with dopamine replacement therapy in Parkinson's disease;
The use of the above combination, in the manufacture of a medicament for the treatment of cognitive function impairment associated with Parkinson's disease

### BRIEF DESCRIPTION OF DRAWINGS

FIG. 1: Receptor occupancy (%) plotted against plasma concentration (ng/ml) of Compound (I). Vertical line shows the corresponding estimated EC₅₀.

FIG. 2: Brain and plasma PK of Compound (I) (upper panel), pitolisant (middle panel) and bavisant (lower panel) after oral administration of 10 mg/kg in rats.

FIG. 3: Time-course receptor occupancy studies of Compound (I) (upper panel), pitolisant (middle panel) and bavisant (lower panel) after oral administration of 10 mg/kg [Compound (I) and bavisant] or 300 mg/kg (pitolisant) in rats.

FIG. 4: tMeHA time-course studies after oral administration of 10 mg/kg of Compound (I) (upper panel), pitolisant (middle panel) and bavisant (lower panel) in rats. The statistical significance of drug vs. vehicle group was analyzed using two-way ANOVA analysis with repeated measures (* p<0.05, *** p<0.01).

### DETAILED DESCRIPTION OF THE INVENTION

It has been found that Compound (I) may be an ideal candidate in the treatment of excessive daytime sleepiness (EDS) associated with PD having therapeutic advantages, such as one or more of the following:
i) it reduces excessive daytime sleepiness (i.e. improves wakefulness), for example, it decreases excessive daytime sleepiness compared to placebo;
ii) it improves (e.g. decreases) subjective sleepiness, for example, it improves Epworth Sleepiness Scale (ESS; Johns, M. W., Sleep, 1991, 14, 540-545) score compared to placebo (e.g. decrease in 3 points from baseline);
iii) it improves objective sleepiness (e.g. improvement in frequency, duration or intensity), for example, it increases sleep latency, e.g. as measured by the Maintenance of Wakefulness Test (MWT) or as measured by the Multiple Sleep Latency Test (MSLT) [e.g. in Littner et al, Sleep, 2005, 28 (1), 113-121], compared to placebo (e.g. at least 0.5 minute increase of sleep latency compared to placebo);
iv) it improves (e.g. decreases) clinical impression of sleepiness, for example, as assessed from Clinical Global Impression scale (CGI; see for example, Guy 1976) score of overall sleepiness compared to placebo;
v) it reduces excessive daytime sleepiness (i.e. improves wakefulness) without affecting nocturnal sleep [e.g. without causing insomnia, for example, as measured by sleep diary data or polysomnography (PSG) measurements (see for example Berry et al 2016)] e.g. compared to placebo;
vi) it reduces excessive daytime sleepiness without affecting nocturnal sleep (e.g. without causing insomnia) compared to other therapeutic agent/s [e.g. pitolisant, bavisant, modafinil, armodafinil or JZP-110 (solriamfetol)];
vii) it improves cognitive function, for example, it improves one or more of the cognitive domains selected from the group consisting of learning, psychomotor function, attention, sustained attention, working memory, episodic memory and executive function [e.g. as measured by the Symbol Digit Modalities Test (SDMT; see for example Smith, 1968) and computerized tests (see for example Cho, et al 2011 or Grove, et al 2014)], compared to placebo;
viii) it reduces fatigue [e.g. as measured by Fatigue Severity Scale (FSS) score, for example, in Archives of Neurology, 1989; 46:1121-1123 or https://www.healthywomen.org/sites/default/files/FatigueSeverityScale.pdf], for example, compared to placebo; or
ix) it has a favorable safety profile, such as a favorable profile in relation to skin reactions, psychiatric adverse events (e.g. no increase or occurrence of depression) or cardiovascular adverse events (e.g. blood pressure, heart rate, electrocardiography parameters); for example, it has a better safety profile compared to other therapeutic agent/s (e.g. pitolisant, bavisant modafinil, armodafinil, or JZP-110 (solriamfetol)].

Embodiments of the present invention are:

### Embodiments (a)

Embodiment 1a: Compound (I), or pharmaceutically acceptable salt thereof, for use in promoting wakefulness in a Parkinson's disease patient.

Embodiment 2a: Compound (I), or pharmaceutically acceptable salt thereof, for use in the treatment of cognitive function impairment associated with Parkinson's disease; such as learning impairment, psychomotor function impairment, attention impairment, sustained attention impairment, working memory impairment, episodic memory impairment and executive function impairment, which are each associated with Parkinson's disease; in particular attention associated with Parkinson's disease.

Embodiment 3a: Compound (I), or pharmaceutically acceptable salt thereof, for use in the treatment of excessive daytime sleepiness associated with Parkinson's disease.

Embodiment 4a: Compound (I), or pharmaceutically acceptable salt thereof, for use in the treatment of excessive daytime sleepiness associated with dopamine replacement therapy in Parkinson's disease.

Embodiment 5a: Compound (I), or pharmaceutically acceptable salt thereof, for use according to any one of embodiments 1a to 4a, wherein Compound (I), or pharmaceutically acceptable salt thereof, is administered in the form of a pharmaceutical composition further comprising at least one pharmaceutically acceptable excipient.

Embodiment 6a: Compound (I), or pharmaceutically acceptable salt thereof, for use according to any one of embodiments 1a to 4a, wherein Compound (I), or pharmaceutically acceptable salt thereof, is administered in combination with one or more further pharmaceutical active ingredient.

Embodiment 7a: Compound (I), or pharmaceutically acceptable salt thereof, for use according to embodiment 6a, wherein the further pharmaceutical active ingredient is a wakefulness-promoting agent.

Embodiment 8a: Compound (I), or pharmaceutically acceptable salt thereof, for use according to embodiment 6a, wherein the further pharmaceutical active ingredient(s) is selected from the group consisting of levodopa; the combination of levodopa and pergolide; the combination of levodopa and cabergoline; the combination of levodopa and ropinirole; the combination of levodopa and carbidopa; the combination of levodopa and entacapone; the combination of levodopa and benserazide; and the combination of levodopa and pramipexole; or pharmaceutically acceptable salts thereof.

Embodiment 9a: Compound (I), or pharmaceutically acceptable salt thereof, for use according to any one of embodiments 1a to 8a, wherein the use is combined with psychological therapy or behavioral therapy, in particular behavioral therapy, such as cognitive behavioral therapy focused on sleep hygiene rules (e.g. wherein the behavioral therapy is computer-assisted).

Embodiment 10a: Compound (I), or pharmaceutically acceptable salt thereof, for use according to any one of embodiments 3a to 9a, wherein excessive daytime sleepiness coexists with one or more sleep disorders associated with Parkinson's disease, such as rapid eye movement, e.g., rapid eye movement sleep behavior disorder.

Embodiment 11a: Compound (I), or pharmaceutically acceptable salt thereof, for use according to any one of embodiments 1a to 10a, wherein Compound (I), is administered in an amount of from 0.1 mg/day to 50 mg/day, in particular of from 1 mg/day to 20 mg/day, such as 5 mg/day, 10 mg/day or 20 mg/day, in particular 10 mg/day.

Embodiment 12a: Compound (I), or pharmaceutically acceptable salt thereof, for use according to any one of embodiments 1a to 11a, wherein Compound (I), or pharmaceutically acceptable salt thereof, is administered orally.

Embodiment 13a: Compound (I), or pharmaceutically acceptable salt thereof, for use according to any one of embodiments 1a to 12a, wherein Parkinson's disease coexists with a psychiatric disorder, such as depression, anxiety or psychosis.

Embodiment 14a: Compound (I), or pharmaceutically acceptable salt thereof, for use according to any one of embodiments 1a to 13a, wherein Parkinson's disease is early-stage of Parkinson's disease, mid-stage Parkinson's or advanced-stage of Parkinson's disease, in particular advanced-stage of Parkinson's disease.

Embodiment 15a: A combination comprising Compound (I), or pharmaceutically acceptable salt thereof, and at least one active ingredient selected from the group consisting of levodopa, the combination of levodopa and pergolide, the combination of levodopa and cabergoline, the combination of levodopa and ropinirole, the combination of levodopa and carbidopa, the combination of levodopa and entacapone, the combination of levodopa and benserazide, the combination of levodopa and pramipexole, amantadine, selegiline, rasagiline, entacapone, ramelteon, melatonin, zolpidem, eszopiclone, zopiclone, brotizolam, trazodone, doxepin, darifenacin, solifenacin, tolterodine, pregabalin, gabapentin, enacarbil, paroxetine, donepezil, rivastigmine, desipramine, carbamazepine, clonazepam, lorazepam, triazolam, temazepam, flurazepam, cabergoline, rotigotine, suvorexant, pergolide, pramipexole, cabergoline, ropinirole, carbidopa, benserazide, clozapine, quetiapine, primavanesrin, duloxetine, mirtazapine, nortriptyline, venlafaxine, modafinil, armodafinil, caffeine, methylphenidate, dextroamphetamine and sodium oxybate; or pharmaceutically acceptable salts thereof; in particular modafinil, armodafinil, caffeine, methylphenidate, dextroamphetamine, alprazolam, solriamfetol, and sodium oxybate; or pharmaceutically acceptable salts thereof.

Embodiment 16a: A combination comprising Compound (I), or pharmaceutically acceptable salt thereof, and at least one active ingredient selected from the group consisting of modafinil, armodafinil, caffeine, methylphenidate, dextroamphetamine, solriamfetol and sodium oxybate, or pharmaceutically acceptable salts thereof; in particular, solriamfetol, modafinil or armodafinil, or pharmaceutically acceptable salts thereof.

### Embodiments (b)

Embodiment 1b: A pharmaceutical composition comprising Compound (I), or pharmaceutically acceptable salt thereof, and at least one pharmaceutically acceptable excipient, for use in promoting wakefulness in a Parkinson's disease patient.

Embodiment 2b: A pharmaceutical composition comprising Compound (I), or pharmaceutically acceptable salt thereof, and at least one pharmaceutically acceptable excipient, for use in the treatment of cognitive function impairment associated with Parkinson's disease; such as learning impairment, psychomotor function impairment, attention impairment, sustained attention impairment, working memory impairment, episodic memory impairment and executive function impairment, which are each associated with Parkinson's disease; in particular attention associated with Parkinson's disease.

Embodiment 3b: A pharmaceutical composition comprising Compound (I), or pharmaceutically acceptable salt thereof, and at least one pharmaceutically acceptable excipient, for use in the treatment of excessive daytime sleepiness associated with Parkinson's disease.

Embodiment 4b: A pharmaceutical composition comprising Compound (I), or pharmaceutically acceptable salt thereof, and at least one pharmaceutically acceptable excipient, for use in the treatment of excessive daytime sleepiness associated with dopamine replacement therapy in Parkinson's disease.

Embodiment 5b: A pharmaceutical composition comprising Compound (I), or pharmaceutically acceptable salt thereof, and at least one pharmaceutically acceptable excipient, for use according to any one of embodiments 1 b to 4b, wherein Compound (I), or pharmaceutically acceptable salt thereof, is administered in combination with one or more further pharmaceutical active ingredient.

Embodiment 6b: A pharmaceutical composition comprising Compound (I), or pharmaceutically acceptable salt thereof, and at least one pharmaceutically acceptable excipient, for use according to embodiment 5b, wherein the further pharmaceutical active ingredient is a wakefulness-promoting agent.

Embodiment 7b: A pharmaceutical composition comprising Compound (I), or pharmaceutically acceptable salt thereof, and at least one pharmaceutically acceptable excipient, for use according to embodiment 5b, wherein the further pharmaceutical active ingredient(s) is selected from the group consisting of levodopa; the combination of levodopa and pergolide; the combination of levodopa and cabergoline; the combination of levodopa and ropinirole; the combination of levodopa and carbidopa; the combination of levodopa and entacapone; the combination of levodopa and benserazide; and the combination of levodopa and pramipexole; or pharmaceutically acceptable salts thereof.

Embodiment 8b: A pharmaceutical composition comprising Compound (I), or pharmaceutically acceptable salt thereof, and at least one pharmaceutically acceptable excipient, for use according to any one of embodiments 1 b to 7b, wherein the use is combined with psychological therapy or behavioral therapy, in particular behavioral therapy, such as cognitive behavioral therapy focused on sleep hygiene rules (e.g. wherein the behavioral therapy is computer-assisted).

Embodiment 9b: A pharmaceutical composition comprising Compound (I), or pharmaceutically acceptable salt thereof, and at least one pharmaceutically acceptable excipient, for use according to any one of embodiments 3b to 8b, wherein excessive daytime sleepiness coexists with one or more sleep disorders associated with Parkinson's disease, such as rapid eye movement, e.g., rapid eye movement sleep behavior disorder.

Embodiment 10b: A pharmaceutical composition comprising Compound (I), or pharmaceutically acceptable salt thereof, and at least one pharmaceutically acceptable excipient, for use according to any one of embodiments 1 b to 9b, wherein Compound (I), is administered in an amount of from 0.1 mg/day to 50 mg/day, in particular of from 1 mg/day to 20 mg/day, such as 5 mg/day, 10 mg/day or 20 mg/day, in particular 10 mg/day.

Embodiment 11b: A pharmaceutical composition comprising Compound (I), or pharmaceutically acceptable salt thereof, and at least one pharmaceutically acceptable excipient, for use according to any one of embodiments 1 b to 10b, wherein Compound (I), or pharmaceutically acceptable salt thereof, is administered orally.

Embodiment 12b: A pharmaceutical composition comprising Compound (I), or pharmaceutically acceptable salt thereof, and at least one pharmaceutically acceptable excipient, for use according to any one of embodiments 1b to 11b, wherein Parkinson's disease coexists with a psychiatric disorder, such as depression, anxiety or psychosis.

Embodiment 13b: A pharmaceutical composition comprising Compound (I), or pharmaceutically acceptable salt thereof, and at least one pharmaceutically acceptable excipient, for use according to any one of embodiments 1 b to 12b, wherein Parkinson's disease is early-stage of Parkinson's disease, mid-stage Parkinson's or advanced-stage of Parkinson's disease, in particular advanced-stage of Parkinson's disease.

### Embodiments (c)

Embodiment 1c: A pharmaceutical combination comprising Compound (I), or pharmaceutically acceptable salt thereof, and at least one further pharmaceutical active ingredient, for use in promoting wakefulness in a Parkinson's disease patient.

Embodiment 2c: A pharmaceutical combination comprising Compound (I), or pharmaceutically acceptable salt thereof, and at least one further pharmaceutical active ingredient, for use in the treatment of cognitive function impairment associated with Parkinson's disease; such as learning impairment, psychomotor function impairment, attention impairment, sustained attention impairment, working memory impairment, episodic memory impairment and executive function impairment, which are each associated with Parkinson's disease; in particular attention associated with Parkinson's disease.

Embodiment 3c: A pharmaceutical combination comprising Compound (I), or pharmaceutically acceptable salt thereof, and at least one further pharmaceutical active ingredient, for use in the treatment of excessive daytime sleepiness associated with Parkinson's disease.

Embodiment 4c: A pharmaceutical combination comprising Compound (I), or pharmaceutically acceptable salt thereof, and at least one further pharmaceutical active ingredient, for use in the treatment of excessive daytime sleepiness associated with dopamine replacement therapy in Parkinson's disease.

Embodiment 5c: A pharmaceutical combination comprising Compound (I), or pharmaceutically acceptable salt thereof, and at least one further pharmaceutical active ingredient, for use according to any one of embodiments 1c to 4c, wherein the further pharmaceutical active ingredient is a wakefulness-promoting agent.

Embodiment 6c: A pharmaceutical combination comprising Compound (I), or pharmaceutically acceptable salt thereof, and at least one further pharmaceutical active ingredient, for use according to any one of embodiments 1c to 4c, wherein the further pharmaceutical active ingredient(s) is selected from the group consisting of levodopa; the combination of levodopa and pergolide; the combination of levodopa and cabergoline; the combination of levodopa and ropinirole; the combination of levodopa and carbidopa; the combination of levodopa and entacapone; the combination of levodopa and benserazide; and the combination of levodopa and pramipexole; or pharmaceutically acceptable salts thereof.

Embodiment 7c: A pharmaceutical combination comprising Compound (I), or pharmaceutically acceptable salt thereof, and at least one further pharmaceutical active ingredient, for use according to any one of embodiments 1c to 6c, wherein the use is combined psychological therapy or behavioral therapy, in particular behavioral therapy, such as cognitive behavioral therapy focused on sleep hygiene rules (e.g. wherein the behavioral therapy is computer-assisted).

Embodiment 8c: A pharmaceutical combination comprising Compound (I), or pharmaceutically acceptable salt thereof, and at least one further pharmaceutical active ingredient, for use according to any one of embodiments 3c to 7c, wherein excessive daytime sleepiness coexists with one or more sleep disorders associated with Parkinson's disease, such as rapid eye movement, e.g., rapid eye movement sleep behavior disorder.

Embodiment 9c: A pharmaceutical combination comprising Compound (I), or pharmaceutically acceptable salt thereof, and at least one further pharmaceutical active ingredient, for use according to any one of embodiments 1c to 8c, wherein Compound (I), is administered in an amount of from 0.1 mg/day to 50 mg/day, in particular of from 1 mg/day to 20 mg/day, such as 5 mg/day, 10 mg/day or 20 mg/day, in particular 10 mg/day.

Embodiment 10c: A pharmaceutical combination comprising Compound (I), or pharmaceutically acceptable salt thereof, and at least one further pharmaceutical active ingredient, for use according to any one of embodiments 1c to 9c, wherein Compound (I), or pharmaceutically acceptable salt thereof, is administered orally.

Embodiment 11c: A pharmaceutical combination comprising Compound (I), or pharmaceutically acceptable salt thereof, and at least one further pharmaceutical active ingredient, for use according to any one of embodiments 1c to 10c, wherein Parkinson's disease coexists with a psychiatric disorder, such as depression, anxiety or psychosis.

Embodiment 12c: A pharmaceutical combination comprising Compound (I), or pharmaceutically acceptable salt thereof, and at least one further pharmaceutical active ingredient, for use according to any one of embodiments 1c to 11c, wherein Parkinson's disease is early-stage of Parkinson's disease, mid-stage Parkinson's or advanced-stage of Parkinson's disease, in particular advanced-stage of Parkinson's disease.

### Embodiments (d)

Embodiment 1d: A method of treatment for promoting wakefulness in a Parkinson's disease subject, in need thereof, comprising administering to said subject an effective amount of Compound (I), or pharmaceutically acceptable salt thereof.

Embodiment 2d: A method for treating cognitive function impairment associated with Parkinson's disease, in a subject, in need thereof, comprising administering to said subject an effective amount of Compound (I), or pharmaceutically acceptable salt thereof, in particular wherein cognitive function comprises cognitive domains selected from the group consisting of learning impairment, psychomotor function impairment, attention impairment, sustained attention impairment, working memory impairment, episodic memory impairment and executive function impairment, which are each associated with Parkinson's disease; in particular attention associated with Parkinson's disease.

Embodiment 3d: A method for treating excessive daytime sleepiness associated with Parkinson's disease, in a subject, in need thereof, comprising administering to said subject an effective amount of Compound (I), or pharmaceutically acceptable salt thereof.

Embodiment 4d: A method for treating excessive daytime sleepiness associated with dopamine replacement therapy in Parkinson's disease, in a subject, in need thereof, comprising administering to said subject an effective amount of Compound (I), or pharmaceutically acceptable salt thereof.

Embodiment 5d: A method according to any one of embodiments 1d to 4d, wherein Compound (I), or pharmaceutically acceptable salt thereof, is administered in the form of a pharmaceutical composition further comprising at least one pharmaceutically acceptable excipient.

Embodiment 6d: A method according to any one of embodiments 1d to 4d, wherein Compound (I), or pharmaceutically acceptable salt thereof, is administered in combination with one or more further pharmaceutical active ingredient.

Embodiment 7d: A method according to embodiment 6d, wherein the further pharmaceutical active ingredient is a wakefulness-promoting agent.

Embodiment 8d: A method according to embodiment 6d, wherein the further pharmaceutical active ingredient(s) is selected from the group consisting of levodopa; the combination of levodopa and pergolide; the combination of levodopa and cabergoline; the combination of levodopa and ropinirole; the combination of levodopa and carbidopa; the combination of levodopa and entacapone; the combination of levodopa and benserazide; and the combination of levodopa and pramipexole; or pharmaceutically acceptable salts thereof.

Embodiment 9d: A method according to any one of embodiments 1d to 8d, wherein the method is combined with psychological therapy or behavioral therapy, in particular behavioral therapy, such as cognitive behavioral therapy focused on sleep hygiene rules (e.g. wherein the behavioral therapy is computer-assisted).

Embodiment 10d: A method according to any one of embodiments 3d to 9d, wherein excessive daytime sleepiness coexists with one or more sleep disorders associated with Parkinson's disease, such as rapid eye movement, e.g., rapid eye movement sleep behavior disorder.

Embodiment 11d: A method according to any one of embodiments 1d to 10d, wherein Compound (I), is administered in an amount of from 0.1 mg/day to 50 mg/day, in particular of from 1 mg/day to 20 mg/day, such as 5 mg/day, 10 mg/day or 20 mg/day, in particular 10 mg/day.

Embodiment 12d: A method according to any one of embodiments 1d to 11d, wherein Compound (I), or pharmaceutically acceptable salt thereof, is administered orally.

Embodiment 13d: A method according to any one of embodiments 1d to 12d, wherein Parkinson's disease coexists with a psychiatric disorder, such as depression, anxiety or psychosis.

Embodiment 14d: A method according to any one of embodiments 1d to 13d, wherein Parkinson's disease is early-stage of Parkinson's disease, mid-stage Parkinson's or advanced-stage of Parkinson's disease, in particular advanced-stage of Parkinson's disease.

### Embodiments (e)

Embodiment 1e: A method of treatment for promoting wakefulness in a Parkinson's disease subject, in need thereof, comprising administering to said subject a pharmaceutical composition comprising an effective amount of Compound (I), or pharmaceutically acceptable salt thereof, and at least one pharmaceutically acceptable excipient.

Embodiment 2e: A method for treating cognitive function impairment associated with Parkinson's disease, in a subject, in need thereof, comprising administering to said subject a pharmaceutical composition comprising an effective amount of Compound (I), or pharmaceutically acceptable salt thereof, and at least one pharmaceutically acceptable excipient, in particular wherein cognitive function comprises cognitive domains selected from the group consisting of learning impairment, psychomotor function impairment, attention impairment, sustained attention impairment, working memory impairment, episodic memory impairment and executive function impairment, which are each associated with Parkinson's disease; in particular attention associated with Parkinson's disease.

Embodiment 3e: A method for treating excessive daytime sleepiness associated with Parkinson's disease, in a subject, in need thereof, comprising administering to said subject a pharmaceutical composition comprising an effective amount of Compound (I), or pharmaceutically acceptable salt thereof, and at least one pharmaceutically acceptable excipient.

Embodiment 4e: A method for treating excessive daytime sleepiness associated with dopamine replacement therapy in Parkinson's disease, in a subject, in need thereof, comprising administering to said subject a pharmaceutical composition comprising an effective amount of Compound (I), or pharmaceutically acceptable salt thereof, and at least one pharmaceutically acceptable excipient.

Embodiment 5e: A method according to any one of embodiments 1e to 4e, wherein Compound (I), or pharmaceutically acceptable salt thereof, is administered in combination with one or more further pharmaceutical active ingredient.

Embodiment 6e: A method according to embodiment 5e, wherein the further pharmaceutical active ingredient is a wakefulness-promoting agent.

Embodiment 7e: A method according to embodiment 5e, wherein the further pharmaceutical active ingredient(s) is selected from the group consisting of levodopa; the combination of levodopa and pergolide; the combination of levodopa and cabergoline; the combination of levodopa and ropinirole; the combination of levodopa and carbidopa; the combination of levodopa and entacapone; the combination of levodopa and benserazide; and the combination of levodopa and pramipexole; or pharmaceutically acceptable salts thereof.

Embodiment 8e: A method according to any one of embodiments 1e to 7e, wherein the method is combined with psychological therapy or behavioral therapy, in particular behavioral therapy, such as cognitive behavioral therapy focused on sleep hygiene rules (e.g. wherein the behavioral therapy is computer-assisted).

Embodiment 9e: A method according to any one of embodiments 3e to 8e, wherein excessive daytime sleepiness coexists with one or more sleep disorders associated with Parkinson's disease, such as rapid eye movement, e.g., rapid eye movement sleep behavior disorder.

Embodiment 10e: A method according to any one of embodiments 1e to 9e, wherein Compound (I), is administered in an amount of from 0.1 mg/day to 50 mg/day, in particular of from 1 mg/day to 20 mg/day, such as 5 mg/day, 10 mg/day or 20 mg/day, in particular 10 mg/day.

Embodiment 11e: A method according to any one of embodiments 1e to 10e, wherein Compound (I), or pharmaceutically acceptable salt thereof, is administered orally.

Embodiment 12e: A method according to any one of embodiments 1e to 11e, wherein Parkinson's disease coexists with a psychiatric disorder, such as depression, anxiety or psychosis.

Embodiment 13e: A method according to any one of embodiments 1e to 12e, wherein Parkinson's disease is early-stage of Parkinson's disease, mid-stage Parkinson's or advanced-stage of Parkinson's disease, in particular advanced-stage of Parkinson's disease.

### Embodiments (f)

Embodiment 1f: A method of treatment for promoting wakefulness in a Parkinson's disease subject, in need thereof, comprising administering to said subject a pharmaceutical combination comprising an effective amount of Compound (I), or pharmaceutically acceptable salt thereof, and at least one further pharmaceutical active ingredient.

Embodiment 2f: A method for treating cognitive function impairment associated with Parkinson's disease, in a subject, in need thereof, comprising administering to said subject a pharmaceutical combination comprising an effective amount of Compound (I), or pharmaceutically acceptable salt thereof, and at least one further pharmaceutical active ingredient, in particular wherein cognitive function comprises cognitive domains selected from the group consisting of learning impairment, psychomotor function impairment, attention impairment, sustained attention impairment, working memory impairment, episodic memory impairment and executive function impairment, which are each associated with Parkinson's disease; in particular attention associated with Parkinson's disease.

Embodiment 3f: A method for treating excessive daytime sleepiness associated with Parkinson's disease, in a subject, in need thereof, comprising administering to said subject a pharmaceutical combination comprising an effective amount of Compound (I), or pharmaceutically acceptable salt thereof, and at least one further pharmaceutical active ingredient.

Embodiment 4f: A method for treating excessive daytime sleepiness associated with dopamine replacement therapy in Parkinson's disease, in a subject, in need thereof, comprising administering to said subject a pharmaceutical combination comprising an effective amount of Compound (I), or pharmaceutically acceptable salt thereof, and at least one further pharmaceutical active ingredient.

Embodiment 5f: A method according to any one of embodiments 1f to 4f, wherein the further pharmaceutical active ingredient is a wakefulness-promoting agent.

Embodiment 6f: A method according to any one of embodiments 1f to 4f, wherein the further pharmaceutical active ingredient(s) is selected from the group consisting of levodopa; the combination of levodopa and pergolide; the combination of levodopa and cabergoline; the combination of levodopa and ropinirole; the combination of levodopa and carbidopa; the combination of levodopa and entacapone; the combination of levodopa and benserazide; and the combination of levodopa and pramipexole; or pharmaceutically acceptable salts thereof.

Embodiment 7f: A method according to any one of embodiments 1f to 6f, wherein the method is combined with psychological therapy or behavioral therapy, in particular behavioral therapy, such as cognitive behavioral therapy focused on sleep hygiene rules (e.g. wherein the behavioral therapy is computer-assisted).

Embodiment 8f: A method according to any one of embodiments 3f to 7f, wherein excessive daytime sleepiness coexists with one or more sleep disorders associated with Parkinson's disease, such as rapid eye movement, e.g., rapid eye movement sleep behavior disorder.

Embodiment 9f: A method according to any one of embodiments 1f to 8f, wherein Compound (I), is administered in an amount of from 0.1 mg/day to 50 mg/day, in particular of from 1 mg/day to 20 mg/day, such as 5 mg/day, 10 mg/day or 20 mg/day, in particular 10 mg/day.

Embodiment 10f: A method according to any one of embodiments 1f to 9f, wherein Compound (I), or pharmaceutically acceptable salt thereof, is administered orally.

Embodiment 11f: A method according to any one of embodiments 1f to 10f, wherein Parkinson's disease coexists with a psychiatric disorder, such as depression, anxiety or psychosis.

Embodiment 12f: A method according to any one of embodiments 1f to 11f, wherein Parkinson's disease is early-stage of Parkinson's disease, mid-stage Parkinson's or advanced-stage of Parkinson's disease, in particular advanced-stage of Parkinson's disease.

### Embodiments (g)

Embodiment 1g: Use of Compound (I), or pharmaceutically acceptable salt thereof, for the manufacture of a medicament for a treatment promoting wakefulness in a Parkinson's disease patient.

Embodiment 2g: Use of Compound (I), or pharmaceutically acceptable salt thereof, for the manufacture of a medicament for the treatment of cognitive function impairment associated with Parkinson's disease; such as learning impairment, psychomotor function impairment, attention impairment, sustained attention impairment, working memory impairment, episodic memory impairment and executive function impairment, which are each associated with Parkinson's disease; in particular attention associated with Parkinson's disease.

Embodiment 3g: Use of Compound (I), or pharmaceutically acceptable salt thereof, for the manufacture of a medicament for the treatment of excessive daytime sleepiness associated with Parkinson's disease.

Embodiment 4g: Use of Compound (I), or pharmaceutically acceptable salt thereof, for the manufacture of a medicament for the treatment of excessive daytime sleepiness associated with dopamine replacement therapy in Parkinson's disease.

Embodiment 5g: Use of Compound (I), or pharmaceutically acceptable salt thereof, for the manufacture of a medicament according to any one of embodiments 1g to 4g, wherein Compound (I), or pharmaceutically acceptable salt thereof, is administered in the form of a pharmaceutical composition further comprising at least one pharmaceutically acceptable excipient.

Embodiment 6g: Use of Compound (I), or pharmaceutically acceptable salt thereof, for the manufacture of a medicament according to any one of embodiments 1g to 4g, wherein Compound (I), or pharmaceutically acceptable salt thereof, is administered in combination with one or more further pharmaceutical active ingredient.

Embodiment 7g: Use of Compound (I), or pharmaceutically acceptable salt thereof, for the manufacture of a medicament according to embodiment 6g, wherein the further pharmaceutical active ingredient is a wakefulness-promoting agent.

Embodiment 8g: Use of Compound (I), or pharmaceutically acceptable salt thereof, for the manufacture of a medicament according to embodiment 6g, wherein the further pharmaceutical active ingredient(s) is selected from the group consisting of levodopa; the combination of levodopa and pergolide; the combination of levodopa and cabergoline; the combination of levodopa and ropinirole; the combination of levodopa and carbidopa; the combination of levodopa and entacapone; the combination of levodopa and benserazide; and the combination of levodopa and pramipexole; or pharmaceutically acceptable salts thereof.

Embodiment 9g: Use of Compound (I), or pharmaceutically acceptable salt thereof, for the manufacture of a medicament according to any one of embodiments 1g to 8g, wherein the use is combined with psychological therapy or behavioral therapy, in particular behavioral therapy, such as cognitive behavioral therapy focused on sleep hygiene rules (e.g. wherein the behavioral therapy is computer-assisted).

Embodiment 10g: Use of Compound (I), or pharmaceutically acceptable salt thereof, for the manufacture of a medicament according to any one of embodiments 3g to 9g, wherein excessive daytime sleepiness coexists with one or more sleep disorders associated with Parkinson's disease, such as rapid eye movement, e.g., rapid eye movement sleep behavior disorder.

Embodiment 11g: Use of Compound (I), or pharmaceutically acceptable salt thereof, for the manufacture of a medicament according to any one of embodiments 1g to 10g, wherein Compound (I), is administered in an amount of from 0.1 mg/day to 50 mg/day, in particular of from 1 mg/day to 20 mg/day, such as 5 mg/day, 10 mg/day or 20 mg/day, in particular 10 mg/day.

Embodiment 12g: Use of Compound (I), or pharmaceutically acceptable salt thereof, for the manufacture of a medicament according to any one of embodiments 1g to 11g, wherein Compound (I), or pharmaceutically acceptable salt thereof, is administered orally.

Embodiment 13g: Use of Compound (I), or pharmaceutically acceptable salt thereof, for the manufacture of a medicament according to any one of embodiments 1g to 12g, wherein Parkinson's disease coexists with a psychiatric disorder, such as depression, anxiety or psychosis.

Embodiment 14g: Use of Compound (I), or pharmaceutically acceptable salt thereof, for the manufacture of a medicament according to any one of embodiments 1g to 13g, wherein Parkinson's disease is early-stage of Parkinson's disease, mid-stage Parkinson's or advanced-stage of Parkinson's disease, in particular advanced-stage of Parkinson's disease.

### Embodiments (h)

Embodiment 1h: Use of a pharmaceutical composition comprising Compound (I), or pharmaceutically acceptable salt thereof, and at least one pharmaceutically acceptable excipient, for the manufacture of a medicament for a treatment promoting wakefulness in a Parkinson's disease patient.

Embodiment 2h: Use of a pharmaceutical composition comprising Compound (I), or pharmaceutically acceptable salt thereof, and at least one pharmaceutically acceptable excipient, for the manufacture of a medicament for the treatment of cognitive function impairment associated with Parkinson's disease; such as learning impairment, psychomotor function impairment, attention impairment, sustained attention impairment, working memory impairment, episodic memory impairment and executive function impairment, which are each associated with Parkinson's disease; in particular attention associated with Parkinson's disease.

Embodiment 3h: Use of a pharmaceutical composition comprising Compound (I), or pharmaceutically acceptable salt thereof, and at least one pharmaceutically acceptable excipient, for the manufacture of a medicament for the treatment of excessive daytime sleepiness associated with Parkinson's disease.

Embodiment 4h: Use of a pharmaceutical composition comprising Compound (I), or pharmaceutically acceptable salt thereof, and at least one pharmaceutically acceptable excipient, for the manufacture of a medicament for the treatment of excessive daytime sleepiness associated with dopamine replacement therapy in Parkinson's disease.

Embodiment 5h: Use of a pharmaceutical composition comprising Compound (I), or pharmaceutically acceptable salt thereof, and at least one pharmaceutically acceptable excipient, for the manufacture of a medicament according to any one of embodiments 1h to 4h, wherein Compound (I), or pharmaceutically acceptable salt thereof, is administered in combination with one or more further pharmaceutical active ingredient.

Embodiment 6h: Use of a pharmaceutical composition comprising Compound (I), or pharmaceutically acceptable salt thereof, and at least one pharmaceutically acceptable excipient, for the manufacture of a medicament according to embodiment 5h, wherein the further pharmaceutical active ingredient is a wakefulness-promoting agent.

Embodiment 7h: Use of a pharmaceutical composition comprising Compound (I), or pharmaceutically acceptable salt thereof, and at least one pharmaceutically acceptable excipient, for the manufacture of a medicament according to embodiment 5h, wherein the further pharmaceutical active ingredient(s) is selected from the group consisting of levodopa; the combination of levodopa and pergolide; the combination of levodopa and cabergoline; the combination of levodopa and ropinirole; the combination of levodopa and carbidopa; the combination of levodopa and entacapone; the combination of levodopa and benserazide; and the combination of levodopa and pramipexole; or pharmaceutically acceptable salts thereof.

Embodiment 8h: Use of a pharmaceutical composition comprising Compound (I), or pharmaceutically acceptable salt thereof, and at least one pharmaceutically acceptable excipient, for the manufacture of a medicament according to any one of embodiments 1h to 7h, wherein the use is combined with psychological therapy or behavioral therapy, in particular behavioral therapy, such as cognitive behavioral therapy focused on sleep hygiene rules (e.g. wherein the behavioral therapy is computer-assisted).

Embodiment 9h: Use of a pharmaceutical composition comprising Compound (I), or pharmaceutically acceptable salt thereof, and at least one pharmaceutically acceptable excipient, for the manufacture of a medicament according to any one of embodiments 3h to 8h, wherein excessive daytime sleepiness coexists with one or more sleep disorders associated with Parkinson's disease, such as rapid eye movement, e.g., rapid eye movement sleep behavior disorder.

Embodiment 10h: Use of a pharmaceutical composition comprising Compound (I), or pharmaceutically acceptable salt thereof, and at least one pharmaceutically acceptable excipient, for the manufacture of a medicament according to any one of embodiments 1h to 9h, wherein Compound (I), is administered in an amount of from 0.1 mg/day to 50 mg/day, in particular of from 1 mg/day to 20 mg/day, such as 5 mg/day, 10 mg/day or 20 mg/day, in particular 10 mg/day.

Embodiment 11h: Use of a pharmaceutical composition comprising Compound (I), or pharmaceutically acceptable salt thereof, and at least one pharmaceutically acceptable excipient, for the manufacture of a medicament according to any one of embodiments 1h to 10h, wherein Compound (I), or pharmaceutically acceptable salt thereof, is administered orally.

Embodiment 12h: Use of a pharmaceutical composition comprising Compound (I), or pharmaceutically acceptable salt thereof, and at least one pharmaceutically acceptable excipient, for the manufacture of a medicament according to any one of embodiments 1h to 11h, wherein Parkinson's disease coexists with a psychiatric disorder, such as depression, anxiety or psychosis.

Embodiment 13h: Use of a pharmaceutical composition comprising Compound (I), or pharmaceutically acceptable salt thereof, and at least one pharmaceutically acceptable excipient, for the manufacture of a medicament according to any one of embodiments 1h to 12h, wherein Parkinson's disease is early-stage of Parkinson's disease, mid-stage Parkinson's or advanced-stage of Parkinson's disease, in particular advanced-stage of Parkinson's disease.

### Embodiments (j)

Embodiment 1j: Use of a pharmaceutical combination comprising Compound (I), or pharmaceutically acceptable salt thereof, and at least one further pharmaceutical active ingredient, for the manufacture of a medicament for a treatment promoting wakefulness in a Parkinson's disease patient.

Embodiment 2j: Use of a pharmaceutical combination comprising Compound (I), or pharmaceutically acceptable salt thereof, and at least one further pharmaceutical active ingredient, for the manufacture of a medicament for the treatment of cognitive function impairment associated with Parkinson's disease; such as learning impairment, psychomotor function impairment, attention impairment, sustained attention impairment, working memory impairment, episodic memory impairment and executive function impairment, which are each associated with Parkinson's disease; in particular attention associated with Parkinson's disease.

Embodiment 3j: Use of a pharmaceutical combination comprising Compound (I), or pharmaceutically acceptable salt thereof, and at least one further pharmaceutical active ingredient, for the manufacture of a medicament for the treatment of excessive daytime sleepiness associated with Parkinson's disease.

Embodiment 4j: Use of a pharmaceutical combination comprising Compound (I), or pharmaceutically acceptable salt thereof, and at least one further pharmaceutical active ingredient, for the manufacture of a medicament for the treatment of excessive daytime sleepiness associated with dopamine replacement therapy in Parkinson's disease.

Embodiment 5j: Use of a pharmaceutical combination comprising Compound (I), or pharmaceutically acceptable salt thereof, and at least one further pharmaceutical active ingredient, for the manufacture of a medicament according to any one of embodiments 1j to 4j, wherein the further pharmaceutical active ingredient is a wakefulness-promoting agent.

Embodiment 6j: Use of a pharmaceutical combination comprising Compound (I), or pharmaceutically acceptable salt thereof, and at least one further pharmaceutical active ingredient, for the manufacture of a medicament according to any one of embodiments 1j to 4j, wherein the further pharmaceutical active ingredient(s) is selected from the group consisting of levodopa; the combination of levodopa and pergolide; the combination of levodopa and cabergoline; the combination of levodopa and ropinirole; the combination of levodopa and carbidopa; the combination of levodopa and entacapone; the combination of levodopa and benserazide; and the combination of levodopa and pramipexole; or pharmaceutically acceptable salts thereof.

Embodiment 7j: Use of a pharmaceutical combination comprising Compound (I), or pharmaceutically acceptable salt thereof, and at least one further pharmaceutical active ingredient, for the manufacture of a medicament according to any one of embodiments 1j to 6j, wherein the use is combined with psychological therapy or behavioral therapy, in particular behavioral therapy, such as cognitive behavioral therapy focused on sleep hygiene rules (e.g. wherein the behavioral therapy is computer-assisted).

Embodiment 8j: Use of a pharmaceutical combination comprising Compound (I), or pharmaceutically acceptable salt thereof, and at least one further pharmaceutical active ingredient, for the manufacture of a medicament according to any one of embodiments 3j to 7j, wherein excessive daytime sleepiness coexists with one or more sleep disorders associated with Parkinson's disease, such as rapid eye movement, e.g., rapid eye movement sleep behavior disorder.

Embodiment 9j: Use of a pharmaceutical combination comprising Compound (I), or pharmaceutically acceptable salt thereof, and at least one further pharmaceutical active ingredient, for the manufacture of a medicament according to any one of embodiments 1j to 8j, wherein Compound (I), is administered in an amount of from 0.1 mg/day to 50 mg/day, in particular of from 1 mg/day to 20 mg/day, such as 5 mg/day, 10 mg/day or 20 mg/day, in particular 10 mg/day.

Embodiment 10j: Use of a pharmaceutical combination comprising Compound (I), or pharmaceutically acceptable salt thereof, and at least one further pharmaceutical active ingredient, for the manufacture of a medicament according to any one of embodiments 1j to 9j, wherein Compound (I), or pharmaceutically acceptable salt thereof, is administered orally.

Embodiment 11j: Use of a pharmaceutical combination comprising Compound (I), or pharmaceutically acceptable salt thereof, and at least one further pharmaceutical active ingredient, for the manufacture of a medicament according to any one of embodiments 1j to 10j, wherein Parkinson's disease coexists with a psychiatric disorder, such as depression, anxiety or psychosis.

Embodiment 12j: Use of a pharmaceutical combination comprising Compound (I), or pharmaceutically acceptable salt thereof, and at least one further pharmaceutical active ingredient, for the manufacture of a medicament according to any one of embodiments 1j to 11j, wherein Parkinson's disease is early-stage of Parkinson's disease, mid-stage Parkinson's or advanced-stage of Parkinson's disease, in particular advanced-stage of Parkinson's disease.

### General Terms

The term “Parkinson's disease” (PD), as used herein, is to be understood, for example, according to the Hoehn and Yahr scale [e.g. in Neurology, 1967, 17 (5): 427-442; or in Mov Disord. 2004 September; 19(9): 1020-8], which is incorporated herein by reference. In one embodiment “PD”, as used herein, refers to “early-stage PD”, “mid-stage PD” and “advanced-stage PD”. As used herein, the term “early-stage PD”, refers to stage 1 and 2, the term “mid-stage PD” refers to stage 3, and the term “advanced-stage PD”, refers to stage 4 and 5; wherein stages are according to the Hoehn and Yahr scale [Neurology, 1967, 17 (5): 427-442]. In one embodiment, PD refers to “early-stage PD”. In another embodiment, PD refers to “mid-stage PD”. In yet another embodiment, PD refers to “advanced-stage PD”.

As used herein, the term “Parkinson's disease patient” or “patient with Parkinson's disease” refers to a patient diagnosed with Parkinson's disease, for example, as defined herein above. In one embodiment, it refers to a Parkinson's disease patient (e.g. as defined herein) with excessive daytime sleepiness (e.g. as defined herein).

The term “excessive daytime sleepiness (EDS) associated with Parkinson's disease”, as used herein, is to be understood, for example, according to ICSD-3 criteria (i.e. according to the International Classification of Sleep Disorders—Third Edition) as defined for hypersomnia due to a medical disorder, such as hypersomnia secondary to Parkinson's disease. ICSD-3 criteria defined for hypersomnia due to a medical disorder, which are incorporated herein by reference, relate to the four diagnostic criteria that need to be met (i.e. all A-D below) for the diagnosis: (A) the patient has daily periods of irrepressible need to sleep or daytime lapses into sleep occurring for at least 3 months, (B) the daytime sleepiness occurs as a consequence of a significant underlying medical or neurological condition, (C) if a multiple sleep latency test [MSLT] is performed, the mean sleep latency is min, and fewer than two sleep onset REM periods (SOREMPs) are observed, and (D) the symptoms are not better explained by another untreated sleep disorder, a mental disorder, or the effects of medications or drugs. Excessive daytime sleepiness may be assessed, for example, as objective sleepiness, with the MSLT (i.e. determining mean (±SD) MSLT latency), as recommended by relevant guidelines [Littner et al, Sleep, 2005, 28 (1), 113-121]. Alternatively, excessive daytime sleepiness may be assessed with the Epworth Sleepiness Scale (ESS) [Sleep, 1991, 14, 540-545; epworthsleepinessscale.com], which is a self-administered 8-items questionnaire with scores interpreted as follows: 0-5 lower normal daytime sleepiness, 6-10 higher normal daytime sleepiness, 11-12 mild excessive daytime sleepiness, 13-15 moderate daytime sleepiness, 16-24 severe daytime sleepiness. In one embodiment, term “excessive daytime sleepiness” (EDS), as used herein, is to be understood as ESS score 13.

The term “dopamine-replacement therapy”, as used herein, refers to the principal symptomatic treatment for PD that is based upon administration of either (i) an agent replacing, or increasing the level of, endogenous dopamine (e.g., levodopa (L-DOPA)), or (ii) a dopamine receptor agonist (e.g., apomorphine).

The term “excessive daytime sleepiness associated with dopamine replacement therapy in Parkinson's disease”, as used herein, is to be understood, for example, according to ICSD-3 criteria (i.e. according to the International Classification of Sleep Disorders—3^{rd} Ed.: American Academy of Sleep Medicine, 2014) as defined for hypersomnia due to a medication or substance, such as hypersomnia due to dopamine replacement therapy. ICSD-3 criteria defined for hypersomnia due to a medication or substance, which are incorporated herein by reference, relate to the three diagnostic criteria that need to be met (i.e. all A-c below) for the diagnosis: (A) the patient has daily periods of irrepressible need to sleep or daytime lapses into sleep, (B) the daytime sleepiness occurs as a consequence of current medication or substance use or withdrawal from a wake-promoting medication or substance, (C) the symptoms are not better explained by another untreated sleep disorder, medical or neurological disorder, or mental disorder.

The term “sleep disorders associated with PD”, as used herein, refers, in particular, to a) parasomnias, such as parasomnias associated or not with rapid eye movement (REM), sleep-related breathing disorders, and sleep-related movement disorders, such as restless leg syndrome (RLS) and periodic limb movement disorder (PLMD)]; and b) sudden sleep attacks.

The term “rapid eye movement (REM) sleep behavior disorder”, “rapid eye movement sleep behavior disorder” or “RDB”, as used herein, is defined, for example, with reference to ICSD-3 criteria (International Classification of Sleep Disorders, 3^{rd} Ed.: American Academy of Sleep Medicine, 2014), which are incorporated herein by reference.

The term “restless leg syndrome”, as used herein, is defined, for example, with reference to ICSD-3 criteria, which are incorporated herein by reference.

The term “sleep-related breathing disorders”, as used herein, is defined, for example, with reference to ICSD-3 criteria, which are incorporated herein by reference.

The term “parasomnias”, as used herein, is defined, for example, with reference to ICSD-3 criteria, which are incorporated herein by reference.

The term “periodic limb movement disorder”, as used herein, is defined, for example, with reference to ICSD-3 criteria, which are incorporated herein by reference.

The term “sleep-related movement disorders”, as used herein, is defined, for example, with reference to ICSD-3 criteria, which are incorporated herein by reference.

The term “sudden sleep attack”, as used herein, refers to episodes of sudden onset of sleep (SOS), often without warning signs.

The term “psychiatric disorder”, as used herein, is to be understood, for example, according to criteria defined in the Diagnostic and Statistical Manual of Mental Disorders 5th Edition (DSM-5), which are incorporated herein by reference.

The term “depression”, as used herein, is to be understood, for example, as depressive disorders according to criteria defined in the DMS-5, which are incorporated herein by reference.

The term “anxiety”, as used herein is to be understood, for example, as anxiety disorders according to criteria defined in the DMS-5, which are incorporated herein by reference.

The term “psychosis”, as used herein, is to be understood, for example, as psychotic disorder due to another medical condition according to criteria defined in the DMS-5, which are incorporated herein by reference.

The term “circadian sleep-wake rhythm”, as used herein, refers to the circadian rhythm (i.e. the “internal body clock” that regulates, for example, sleeping patterns, such as when to sleep and when to wake every 24 hours, wherein the normal circadian clock is set by the light-dark cycle over 24 hr).

The term “wakefulness-promoting agent”, as used herein, refers to an active agent capable of decreasing excessive daytime sleepiness, for example, compared with excessive daytime sleepiness observed without treatment. For example, a wakefulness-promoting agent is selected from the group consisting of modafinil, armodafinil, caffeine, methylphenidate, dextroamphetamine, solriamfetol and sodium oxybate, or pharmaceutically acceptable salts thereof; in particular, solriamfetol, modafinil or armodafinil, or pharmaceutically acceptable salts thereof.

The term “promoting wakefulness”, as used herein, refers to decreasing excessive daytime sleepiness, for example, compared with excessive daytime sleepiness observed without treatment, for example as measured by the Epworth Sleepiness Scale (e.g. decrease in 2 points) or as measured by the Maintenance of Wakefulness Test (e.g. at least 0.5 minute increase of sleep latency). In one embodiment, the term “for use in promoting wakefulness”, as used herein, is to be understood as “for use in a treatment promoting wakefulness” or “for use in a method of treatment promoting wakefulness”. In another embodiment, the term “for a treatment promoting wakefulness”, as used herein, is to be understood as “for a method of treatment promoting wakefulness”.

The term “sleep-inducing agent” refers to a compound capable of inducing sleep and/or improving the patient's quality of sleep.

The term “cognitive function” as used herein refers, for example, to the ability to concentrate, remember things, make decisions, solve problems or think. Cognitive function comprises one or more cognitive domains selected from the group consisting of learning, psychomotor function, attention, sustained attention, working memory, episodic memory, and executive function. In one particular embodiment it comprises one or more cognitive domains selected from the group consisting of psychomotor function, attention, sustained attention, working memory, episodic memory and executive function; more particularly, cognitive domains selected from the group consisting of psychomotor function, attention, sustained attention, working memory, episodic memory and executive function.

The term “cognitive function impairment” refers to a deficit in one or more of the cognitive domains relating to cognitive function, in particular a deficit in one or more cognitive domains selected from the group consisting of learning (i.e. learning impairment), psychomotor function (i.e. psychomotor function impairment), attention (i.e. attention impairment), sustained attention (i.e. sustained attention impairment), working memory (i.e. working memory impairment), episodic memory (i.e. episodic memory impairment) and executive function (i.e. executive function impairment); for example, as measured by the Symbol Digit Modalities Test (SDMT; see for example Smith, 1968) and computerized tests (see for example Cho, et al 2011 or Grove, et al 2014). In one particular embodiment cognitive function impairment refers to attention impairment, sustained attention impairment or psychomotor function impairment. In another particular embodiment it refers to learning impairment, episodic memory impairment, working memory impairment or executive function impairment.

The term “attention” as used herein, refers to, but is not limited to, the ability to selectively concentrate on one aspect of the environment while ignoring other things. It may be measured, for example, by the Identification Test (e.g. https://cogstate.com/cognitive-tests/identification/).

The term “psychological therapy”, as used herein, refers to, but is not limited to, standard counselling sessions, for example once a week, for example focused on

The term “behavioral therapy”, as used herein, refers to, but not limited to, cognitive behavioral therapy (e.g. in Koychev et al, Evid Based Ment Health, 2017, 20(1), 15-20), in particular focused on sleep hygiene rules.

The term “sleep hygiene rules”, as used herein, refers to sleep practices and habits, for example, by following routines (e.g. a set of rules), that promote nighttime sleep quality and daytime alertness (e.g. by having a regular bedtime, by limiting daytime naps to 30 minutes, etc.).

In one embodiment, the term “psychological therapy” or “behavioral therapy” comprises light therapy (e.g. phototherapy that uses visible radiation of from 400 to 760 nm), mindfulness (e.g. body scan meditation) or awareness training (e.g. self-awareness training).

The term “computer-assisted” in the expression “the behavioral therapy is computer-assisted”, as used herein, refers to behavioral therapy comprising the use of electronic tools such as online tools, smartphones, wireless devices or health Apps. In one embodiment, the term “computer-assisted” in the expression “the psychosocial or the behavioral therapy is computer-assisted”, as used herein, is to be understood as “computer-implemented” (i.e. the psychosocial or the behavioral therapy is computer-implemented, i.e., provided by a computerized device, such as a mobile device, for example, selected from the group consisting of a smartphone, a laptop computer, a tablet computer, and a wearable computer [e.g. a smartwatch (such as an Apple Watch, a Samsung Gear smartwatch, a LG G Watch, a Sony smartwatch) or a computerized wristband (i.e. smart wristband); in particular a smartphone]. As used herein, the term “treat”, “treating” or “treatment” in connection to a disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those, which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. The term “alleviating” or “alleviation”, for example in reference to a symptom of a condition, as used herein, refers to reducing at least one of the frequency and amplitude of a symptom of a condition in a patient. In one embodiment, the terms “method for the treatment” or “method for treating”, as used herein, refer to “method to treat”.

As used herein, bid=b.i.d=taken twice (two times) a day, for example taken in the morning and evening (separated by approximately 12 hour intervals).

The term “patient”, as used herein, refers to a subject who is diseased and would benefit from the treatment. The term “elderly patient”, as used herein, refers to a patient sixty-five years of age or older.

As used herein, the term “subject” refers to a mammalian organism, preferably a human being (male or female).

As used herein, a subject is “in need of” a treatment if such subject (patient) would benefit biologically, medically or in quality of life from such treatment.

The term “a therapeutically effective amount” of a compound of the present invention refers to an amount of a compound of the present invention that will elicit the biological or medical response of a subject, for example, ameliorate symptoms, alleviate conditions, etc. The term “pharmaceutical composition” is defined herein to refer to a mixture or solution containing at least one active ingredient or therapeutic agent to be administered to a subject, in order to treat a particular condition (i.e. disease, disorder or condition or at least one of the clinical symptoms thereof) affecting the subject.

The term “pharmaceutical composition” is defined herein to refer to a mixture or solution containing at least one active ingredient or therapeutic agent to be administered to a subject, in order to treat a particular condition (i.e. disease, disorder or condition or at least one of the clinical symptoms thereof) affecting the subject.

As used herein, the term “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 22^{nd} Ed. Mack Printing Company, 2013, pp. 1049-1070). Except insofar as any conventional carrier is incompatible with the active ingredient, its use in the therapeutic or pharmaceutical compositions is contemplated.

The terms “drug”, “active substance”, “active ingredient”, “pharmaceutically active ingredient”, “active agent” or “therapeutic agent” are to be understood as meaning a compound in free form or in the form of a pharmaceutically acceptable salt, in particular compounds of the type specified herein.

The term “combination” or “pharmaceutical combination” refers to either a fixed combination in one unit dosage form (e.g., capsule or tablet), non-fixed combination, or a kit of parts for the combined administration where a compound of the present invention and one or more combination partner (e.g. another drug as specified herein, also referred to as further “pharmaceutical active ingredient”, “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. the compound of the present invention and one or more combination partners, are both administered to a patient simultaneously in the form of a single entity or dosage. The term “non-fixed combination” means that the active ingredients, e.g. a compound of the present invention and one or more combination partners, are both administered to a patient as separate entities either simultaneously or sequentially with no specific time limits, wherein such administration provides therapeutically effective levels of the two compounds in the body of the patient.

The compound of the present invention may be administered separately, by the same or different route of administration, or together in the same pharmaceutical composition as the other agents. In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

In particular, reference to a combination with a further active agent, as used herein (e.g. in any of embodiments herein above, or in any of the claims, herein below), refers, for example, to a combination with at least one further active agent, for example, selected from the group of levodopa, the combination of levodopa and pergolide, the combination of levodopa and cabergoline, the combination of levodopa and ropinirole, the combination of levodopa and carbidopa, the combination of levodopa and entacapone, the combination of levodopa and benserazide, the combination of levodopa and pramipexole, amantadine, selegiline, rasagiline, entacapone, ramelteon, melatonin, zolpidem, eszopiclone, zopiclone, brotizolam, trazodone, doxepin, darifenacin, solifenacin, tolterodine, pregabalin, gabapentin, enacarbil, paroxetine, donepezil, rivastigmine, desipramine, carbamazepine, clonazepam, lorazepam, triazolam, temazepam, flurazepam, cabergoline, rotigotine, suvorexant, pergolide, pramipexole, cabergoline, ropinirole, carbidopa, benserazide, clozapine, quetiapine, primavanesrin, duloxetine, mirtazapine, nortriptyline, venlafaxine, modafinil, armodafinil, caffeine, methylphenidate, dextroamphetamine and sodium oxybate; or pharmaceutically acceptable salts thereof; in particular modafinil, armodafinil, caffeine, methylphenidate, dextroamphetamine, alprazolam, solriamfetol, and sodium oxybate; or pharmaceutically acceptable salts thereof.

As used herein, the term “a,” “an,” “the” and similar terms used in the context of the present invention (especially in the context of the claims) are to be construed to cover both the singular and plural unless otherwise indicated herein or clearly contradicted by the context.

The use of any and all examples, or exemplary language (e.g. “such as”) provided herein is intended merely to better illuminate the invention and does not pose a limitation on the scope of the invention otherwise claimed.

As used herein, the compound of the invention, named Compound (I), as used herein above and below, is 1-(1-methyl-6-oxo-1,6-dihydropyridazin-3-yl)piperidin-4-yl 4-cyclobutylpiperazine-1-carboxylate, of formula:

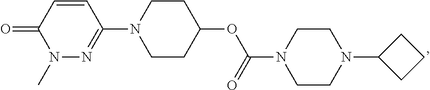 [SMILES: Cn1nc(N2CCC(OC(=O)N3CCN(C4CCC4)CC3)CC2)ccc1=O]

which can be e.g. prepared as described in WO2014/013469, e.g., in Example 1.5. WO2014/013469, which is incorporated herein by reference, also describes its in-vitro biological data, as per pages 40 to 42, as well as solid forms thereof, such as the free form in crystalline form, namely Ex. II. 1.1 (i.e. form A of the free form) and Ex. II. 1.2 (i.e. form B of the free form), as well as salts, for example the citrate salt (i.e. Ex. II. 2.1: form A of the citrate salt; Ex. II. 2.2: form B of the citrate salt), the hydrochloride salt (i.e. Ex. II. 4.1: form A of the hydrochloride salt; Ex. II. 4.2: form B of the hydrochloride salt), the fumarate salt (i.e. Ex. II. 3.1: form A of the fumarate salt; Ex. II. 3.2: form B of the fumarate salt), including preparations thereof. As used herein, 1-(1-methyl-6-oxo-1,6-dihydropyridazin-3-yl)piperidin-4-yl 4-cyclobutylpiperazine-1-carboxylate, or a pharmaceutically acceptable salt thereof, refers in particular to the free form, such as the form A or B of the free form, the citrate salt, such as the form A or B of the citrate salt, the hydrochloride salt, such as the form A or B of the hydrochloride salt, the fumarate salt, such as the form A or B of the fumarate salt. In one embodiment the compound of the invention 1-(1-methyl-6-oxo-1,6-dihydropyridazin-3-yl)piperidin-4-yl 4-cyclobutylpiperazine-1-carboxylate is in the form A of the free form. In another embodiment, compound of the invention 1-(1-methyl-6-oxo-1,6-dihydropyridazin-3-yl)piperidin-4-yl 4-cyclobutylpiperazine-1-carboxylate is in the form B of the free form.

In one embodiment, 1-(1-methyl-6-oxo-1,6-dihydropyridazin-3-yl)piperidin-4-yl 4-cyclobutylpiperazine-1-carboxylate is also intended to represent isotopically labeled forms. Isotopically labeled compounds have structures depicted by the formulas except that one or more atoms are replaced by an atom having a selected atomic mass or mass number. Isotopes that can be incorporated into the compound of the invention include, for example, isotopes of hydrogen, namely the compound of formula:

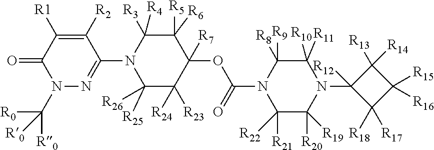

wherein each R₀, R′₀, R″₀, R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅ and R₂₆ is independently selected from H or deuterium; provided that there is at least one deuterium present in the compound. In other embodiments there are multiple deuterium atoms present in the compound. In on embodiment, for example, R0, R′0 and R″0 are deuterium atoms. In another embodiment, for example, R12 is a deuterium atom. In yet another embodiment, for example R1 and R2 are deuterium. In still a further embodiment, for example, R13 to R18 are deuterium atoms.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of the compound of the invention. The concentration of deuterium, may be defined by the isotopic enrichment factor. The term “isotopic enrichment factor” as used herein means the ratio between the isotopic abundance and the natural abundance of a specified isotope. If a substituent in the compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into the compound of the invention include isotopes of hydrogen, other than deuterium, carbon, nitrogen, oxygen, and fluorine such as ³H, ¹¹C, ¹³C, ¹⁴C, ¹⁵N, ¹⁸F respectively. Accordingly, it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. The isotopically-labeled compounds can generally be prepared by conventional techniques known to those skilled in the art or by processes analogous to those described preparation of the compound of the invention by using an appropriate isotopically-labeled reagent in place of the non-labeled reagent previously employed.

As used herein, the terms “free form” or “free forms” refers to the compound in non-salt form, such as the base free form or the acid free form of a respective compound, e.g. the compounds specified herein (e.g. Compound (I) or further pharmaceutical active ingredient, such as a wakefulness-promoting agent, for example, as defined herein).

As used herein, the terms “salt”, “salts” or “salt form” refers to an acid addition or base addition salt of a respective compound, e.g. the compounds specified herein (e.g. Compound (I) or further pharmaceutical active ingredient, such as a wakefulness-promoting agent, for example, as defined herein). “Salts” include in particular “pharmaceutically acceptable salts”. The term “pharmaceutically acceptable salts” refers to salts that retain the biological effectiveness and properties of the compounds and, which typically are not biologically or otherwise undesirable. The compounds, as specified herein (e.g. Compound (I) or further pharmaceutical active ingredient, such as a wakefulness-promoting agent, for example, as defined herein), may be capable of forming acid and/or base salts by virtue of the presence of amino and/or carboxyl groups or groups similar thereto. The compound of the invention is capable of forming acid addition salts by virtue of the presence of amino group similar thereto, such as the citrate salt, hydrochloride salt, fumarate salt, adipate salt, maleate salt or sebacate salt thereof; in particular, the citrate salt, hydrochloride salt and fumarate salt thereof. Thus, as used herein, the term pharmaceutically acceptable salt of 1-(1-methyl-6-oxo-1,6-dihydropyridazin-3-yl)piperidin-4-yl 4-cyclobutylpiperazine-1-carboxylate means a pharmaceutically acceptable acid addition salt of 1-(1-methyl-6-oxo-1,6-dihydropyridazin-3-yl)piperidin-4-yl 4-cyclobutylpiperazine-1-carboxylate.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca, Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 22^{nd} edition, Mack Publishing Company (2013); and in “Handbook of Pharmaceutical Salts: Properties, Selection, and Use” by Stahl and Wermuth (Wiley-VCH, Weinheim, 2011, 2^{nd} edition).

The compounds specified herein (e.g. Compound (I) or further pharmaceutical active ingredient, such as a wakefulness-promoting agent, for example, as defined herein) can be administered by conventional route, in particular orally, such as in the form of tablets or capsules, which can be manufactured according to pharmaceutical techniques as known in the art (for example in “Remington Essentials of Pharmaceutics, 2013, 1^{st} Edition, edited by Linda Felton, published by Pharmaceutical Press 2012, ISBN 978 0 85711 105 0; in particular Chapter 30), wherein pharmaceutical excipients are, for example, as described in “Handbook of Pharmaceutical Excipients, 2012, 7^{th} Edition, edited by Raymond C. Rowe, Paul J. Sheskey, Walter G. Cook and Marian E. Fenton, ISBN 978 0 85711 027 5″.

The pharmaceutical composition or combination of the present invention can be in a unit dosage form (e.g. tablet or capsule) comprising an amount ranging of from 0.1 mg to 50 mg, in particular of from 1 mg to 20 mg, such as 5 mg, 10 mg or 20 mg, in particular 10 mg, of Compound (I) (referring to an amount of the free form of Compound (I), and if a salt thereof is used the amount will be adapted accordingly; in particular Compound (I) is in the free form, such as the form A of the free form or the form B of the free form). For the above-mentioned uses/treatment methods the appropriate dosage may vary depending upon a variety of factors, such as, for example, the age, weight, sex, the route of administration or salt employed. In patients with, for example, of from 50-70 kg body weight, an indicated daily dosage is of from 0.1 mg/day to 50 mg/day, in particular of from 1 mg/day to 20 mg/day, such as 5 mg/day, 10 mg/day or 20 mg/day, in particular 10 mg/day, of Compound (I) [referring to an amount of the free form of Compound (I), and if a salt thereof is used the amount will be adapted accordingly; in particular Compound (I) is in the free form, such as the form A of the free form or the form B of the free form].

### REFERENCES

Smith, A. (1968). The symbol-digit modalities test: a neuropsychologic test of learning and other cerebral disorders. In J. Helmuth (Ed.), Learning disorders (pp. 83-91). Seattle: Special Child Publications.
Guy W (1976) ECDEU Assessment Manual for Psychopharmacology.
Berry R B, Brooks R, Gamaldo C E, Harding S M, Lloyd R M, Marcus C L and Vaughn B V for the American Academy of Sleep Medicine. The AASM Manual for the Scoring of Sleep and Associated Events: Rules, Terminology and Technical Specifications, Version 2.3. www.aasmnet.org. Darien, Ill.: American Academy of Sleep Medicine, 2016.
Cho W, Maruff P, Connell J, et al. (2011) Additive effects of a cholinesterase inhibitor and a histamine inverse agonist on scopolamine deficits in humans. Psychopharmacology (Berl), 218(3):513-24.
Grove R A, Harrington C M, Mahler A, et al. (2014) A randomized, double-blind, placebo-controlled, 16-week study of the H3 receptor antagonist, GSK239512 as a monotherapy in subjects with mild-to-moderate Alzheimer's disease. Curr Alzheimer Res, 11(1):47-58.
Gumenyuk V, Howard R, Roth T, et al. (2014) Sleep loss, circadian mismatch, and abnormalities in reorienting of attention in night workers with shift work disorder. Sleep, 37:545-56.
Gumenyuk V, Roth T and Drake C L (2012) Circadian phase, sleepiness, and light exposure assessment in night workers with and without shift work disorder. Chronobiol Int, 29:928-36.

### Abbreviations

ACN acetonitrile
° C. degree Celcius
EDTA Ethylenediaminetetraacetic acid

### ESS Epworth Sleepiness Scale

FA formaldehyde
g gram(s)
h hour(s)
H₂O water
HPLC high pressure liquid chromatography
HPLC-MS high pressure liquid chromatography-mass spectroscopy

### IACUC Institutional Animal Care and Use Committee

IC₅₀′ inhibitor's concentration which causes 50% inhibition
IV=i.v. intravenous
PO=p.o. by mouth
Kd equilibrium dissociation constant
Kg kilogram
Ki equilibrium inhibitor constant
LCMS liquid chromatography mass spectroscopy
MCP-modeling multiple comparison procedure—modeling
MeOH methanol
min minute(s)
ml=mL milliliter
mM millimolar
mm millimeter
MS mass spectroscopy
ng nanogram
NH₃ ammonia
NH₄OAC ammonium acetate
PK pharmacokinetics
RO receptor occupancy
rpm revolutions per minute
sec second(s)
Tris tris(hydroxymethyl)aminomethane
T½ half-life
PM micromolar
Pm micrometer
v/v volume/volume

### EXAMPLES

### Example 1: Receptor Occupancy (RO) Study

An open-label, adaptive design study was performed in healthy volunteers to characterize regional brain H3 receptor occupancy following single dose of Compound (I) by using positron emission tomography (PET) with the radioligand [¹¹C]MK-8278 (Van Laere, K. J. et al, Journal of Nuclear Medicine, 2014, 55: 65-72). The primary objective of this study was to evaluate the plasma concentration of Compound (I) leading to 50% occupancy of H3 receptors in the brain (EC₅₀). A total of 6 subjects were enrolled into the study and were scanned in three cohorts of two subjects each. Prior to dosing with Compound (I), each subject underwent a baseline PET scan to assess binding of [¹¹C]MK-8278 to H3R in the absence of Compound (I). Subjects subsequently received single oral doses of Compound (I) and then underwent two post-dose PET scans. Subjects of cohort 1 received a single oral dose of 300 mg of Compound (I), corresponding to the single dose maximum tolerated dose (MTD) determined from the Phase I study after single oral dose of Compound (I). These subjects underwent two post-dose PET scans, at 3 hours and 27 hours after dosing. The doses and scan timings for the subsequent cohorts were selected based on interim data analyses following the completion of the preceding cohort(s). Subjects of cohorts 2 and 3 received a single oral dose of 10 mg and 0.3 mg, respectively. Compound (I) was administered as capsules in cohorts 1 and 2, whereas cohort 3 received Compound (I) as an oral solution. In part 3 of the FIH study (Food and formulation effect), no clinically relevant difference in PK was observed after administration of Compound (I) either as a capsule or as an oral solution. The first post-dose PET scan for cohorts 2 and 3 was acquired at 3 hours and the second one at 27 hours and 8 hours after dosing, respectively.

The PET images acquired at baseline displayed the expected heterogeneous signal with the highest volume of distribution (V_{T}) in putamen, followed by caudate, anterior cingulate, most neocortical areas, midbrain and cerebellum. The binding was consistent with both the known distribution of H3 receptors and previous [¹¹C]MK-8278 data (Van Laere, K. J. et al, Journal of Nuclear Medicine, 2014, 55: 65-72). The kinetics of [¹¹C]MK-8278 in brain were analyzed using different modelling approaches, such as the one- and two-tissue compartment (1TC, 2TC) models, multilinear analysis (MA1; Ichise, M. et al., Journal of Cerebral Blood Flow and Metabolism, 2002, 22: 1271-1281) and the Logan graphical method (Logan, J. et al., Journal of Cerebral Blood Flow and Metabolism, 1990, 10(5): 740-747). All methods produced similar results, but the 2TC model was the most appropriate to derive regional V_{T} values. Tissue time-activity curves (TACs) were generated for all scans and were characterized by a peak at around 5-15 minutes post-injection, followed by washout. After administration of Compound (I), regional binding of [¹¹C]MK-8278 was reduced compared to baseline scans, indicating engagement of Compound (I) with the target. The occupancy plot method proposed by Lassen (Lassen, N. A. et al., Journal of Cerebral Blood Flow and Metabolism, 1995, 15 (1): 152-165; Cunningham, V. J. et al., Journal of Cerebral Blood Flow and Metabolism, 2010, 30 (1): 46-50) was applied to estimate whole-brain H3 receptor occupancy for each post-dose PET scan. This method also provided consistent estimates of the non-displaceable component (V_{ND}).

The relationship between plasma concentration of Compound (I) and H3 receptor occupancy was characterized plotting occupancy estimates against corresponding plasma concentration data (measured at the start of each post-dose PET scan). The RO versus concentration was analyzed using the Emax model:

RO=(Emax*Cp)/(Cp+EC₅₀)

Where RO=Receptor occupancy; Emax=maximum receptor occupancy; Cp=plasma concentration of Compound (I) during PET scan; EC₅₀=plasma concentration leading to 50% of maximum change in RO.

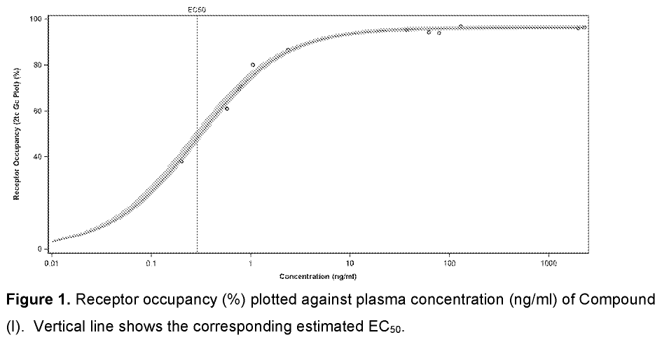

The RO was higher than 95% at 3 hours and 27 hours post-dose in both subjects of the first cohort. RO was similarly high in the second cohort at 3 hours post dose, but decreased at 27 hours post dose. RO values in the third cohort were generally lower than in the other cohorts, particularly at 8 h post dose. A clear relationship was observed in this study between the plasma concentration of Compound (I) and the resulting H3 receptor occupancy estimates, strongly supporting a direct PK-RO relationship. The PK-RO relationship described using an Emax model provided parameters of Emax of 96.1% and EC_{K} of 0.29 ng/mL (FIG. 1).

This study confirmed that Compound (I) binds to H3 receptor in the human brain and allowed to establish the plasma PK-brain RO relationship.

### Example 2: Healthy Volunteers Single Ascending Dose (SAD) and Multiple Ascending Dose (MAD) Study

| | No. of | | |
|---|---|---|---|
| Population | Subjects | | |
| (No. of | exposed to | | |
| enrolled | Compound | | Dose/Frequency/ |
| subjects) | (I)/Placebo | Study Title | Formulation |
| HV (n = 133) | 98 | A first-in-human, randomized, | Part 1 (SAD): 0.3, |
| | Compound | double-blind, | 1, 3, 10, 30, 100, |
| | (I)/35 | placebo-controlled, single and | 300, 800 mg single |
| | placebo | multiple ascending oral dose | dose, capsules |
| | | study, to assess the safety, | Part 2 (MAD): 10, |
| | | tolerability and | 30, 50, 100 mg |
| | | pharmacokinetics of | q.d. dose for 14 |
| | | Compound (I) in healthy | days, capsules |
| | | volunteers | |

Part 1 of the study (SAD) and Part 2 (MAD) assessed the safety, tolerability and PK of increasing single and multiple oral doses of Compound (I) in healthy volunteers, respectively.

### 2.1 Data Used in the PK Model

PK data (drug concentration over time) from Part 1 and Part 2 of the Phase I study were used in the development of the model:
1) Part 1 is the Single Ascending Dose part; 8 different cohorts were used, with each cohort composed of 6 active subjects and 2 placebo subjects. The dose amounts used in the 8 cohorts were 0.3, 1, 3, 10, 30, 100, 300 and 800 mg. Dosing was performed at time equal to 0 hours. Additional data from the Part 3 of the Phase I study (Mechanistic biomarker study, 100 mg single dose) were lumped with this group.
2) Part 2 is the Multiple Ascending Dose part; 4 different cohorts were used, with each cohort composed of 9 active subjects and 3 placebo subjects. The dose amounts used in the 4 cohorts were 10, 30, 50 and 100 mg. Dosing was performed once a day (in the morning) at times equal to 0, 2, 3, 4, 5, 6, 7, 8, 9, 10, 11, 12, 13, 14, 15 days.

No outliers were considered in this work. Data that were below the limit of quantification were excluded from this analysis.

2.2 Data on Night Sleep Duration from the Sleep Diary

Night sleep duration data (total time of night sleep over time) from Part 1 and Part 2 of the Phase I study were used to assess the safety (night sleep disturbance) of Compound (I):
1) Part 1 is the Single Ascending Dose part; 8 different cohorts were used, with each cohort composed of 6 active subjects and 2 placebo subjects. The dose amounts used in the 8 cohorts were 0.3, 1, 3, 10, 30, 100, 300 and 800 mg. Dosing was performed at time equal to 0 hours. Sleep duration data were collected pre-dose and 24 hours post-dosing. Additional data from the Part 3 of the Phase I study (Mechanistic biomarker study, 100 mg single dose) were lumped with this group.
2) Part 2 is the Multiple Ascending Dose part; 4 different cohorts were used, with each cohort composed of 9 active subjects and 3 placebo subjects. The dose amounts used in the 4 cohorts were 10, 30, 50 and 100 mg. Dosing was performed once a day (in the morning) at times equal to 0, 2, 3, 4, 5, 6, 7, 8, 9, 10, 11, 12, 13, 14, 15 days. Sleep duration data were collected at 1, 2, 3, 4, 5, 6, 7, 8, 9, 10, 11, 12, 13, 14, 15 days post-dosing.

### Example 3: Dose Selection for Excessive Daytime Sleepiness in Parkinson's Disease (EDS PD) Study

Data from the Phase I SAD and MAD study (Example 2: PK) as well as data from the PET study RO (Example 1) were used to build a quantitative non-linear mixed effects model that links PK-RO for Compound (I). The model predictions were based on 10,000 simulated subjects.

The RO should be high enough for the H3R inverse agonists to reach full efficacy throughout the desired duration of action; an 80% RO at the maximum drug concentration (Cmax) is required, but higher (90) is preferred (lannone, R. et al., Clinical Pharmacology and Therapeutics, 2010, 88 (6): 831-839). On the other hand, the RO should be low enough after the desired duration of action to avoid night sleep disturbance; although the RO level that results in sleep disturbances is not known, an RO>70% at night may be associated with insomnia (Boström, E. et al., Pharmaceutical Research, 2014, 31: 489-499). Therefore, to minimize sleep disturbances the RO should be as high as possible during the period of duration of action and as low as possible at the time of intended sleep.

In the EDS PD study, the Compound (I) will be administered to the patients in the morning. The administered dose should provide sufficient RO during the expected duration of action (12 hours), while keeping the night sleep disturbance at the minimum.

A 10 mg dose is predicted as the lowest dose providing full efficacy with the least effect on night sleep duration. A 20 mg dose is expected to have similar efficacy to 10 mg but a potentially different safety profile without major tolerability issues (the highest tolerated repeated dose in the healthy volunteer study was 50 mg). The model predicted that a dose of 10 mg will result in a RO 90% for 12 hours post-dosing and 80% for 13-17 hours post-dosing in 90% of the subjects. Given that a dose of 10 mg in FIH study showed a disturbance in night sleep duration comparable to placebo, it is not expected to cause significant night sleep disturbance

### PK Model Description

Modeling Strategy

The analysis was performed using NONMEM VII version 3 (Icon Development Solutions, Ellicott City, Md., USA), utilizing the MODESIM high performance computing environment. Pirana software was used for model editing and model submission to the cluster for running. All model building was performed using the first order conditional estimation with interaction (FOCEi) method.

Structural Model

A two-compartment model with combined zeroth-order and first-order absorption was used to fit the Compound (I) concentrations. The disposition kinetics were modeled using a parameterization involving apparent oral clearance (CL/F), apparent central volume (Vc/F), apparent inter-compartment clearance (Q/F), and apparent peripheral volume (Vp/F). The duration of zero-order absorption (D), a first order absorption rate constant (Kₐ) and an absorption delay parameter (Lag) were used to characterize the absorption process.

Random Effects Model

Between-subject variability in pharmacokinetic parameters Vc/F, CL/F, Vp/F and Kₐ was modeled using multiplicative exponential random effects of the form:

θᵢ=θ×e^{η}^{ᵢ}

where θ is the population typical value of the parameter and ηᵢ denotes the between subject random effect accounting for the i^{th} individual's deviation from the population typical value having mean of zero and variance of ω². Covariance between the parameters Vc/F, CL/F and Vp/F was used.

Residual variability was modeled using a proportional error model:

Yᵢⱼ=Fᵢⱼ×(1+εᵢⱼ)

where Yᵢⱼ denotes the observed concentration for the i^{th} individual at time j. Fᵢⱼ denotes the corresponding predicted concentration based on the pharmacokinetic model. εᵢⱼ denotes the proportional residual random effect, which is assumed to have mean of zero and variance of σ².

Model Parameter Estimates

TABLE 1
Parameter estimates of the PK model

| Parameter (unit) | Estimate (RSE) | |
|---|---|---|
| V_{C}/F (L) | 121 | (3.35%) |
| V_{P}/F (L) | 18.4 | (13.18%) |
| Q/F (L/hr) | 1.26 | (17.35%) |
| CL/F (L/hr) | 22.8 | (4.85%) |
| Kₐ(1/hr) | 2.44 | (15.68%) |
| Lag (hr) | 0.214 | (6.76%) |
| D (hr) | 0.108 | (92.1%) |
| Residual Unexplained Variability (%) | 34.8 | (4.09%) |
| Omega | % CV (RSE) | |
| V_{c}/F | 23.71 | (13.42%) |
| Vₚ/F | 46.48 | (13.69%) |
| CL/F | 40.37 | (7.33%) |
| Kₐ | 145 | (8.59%) |
| Omega correlations | % CV (RSE) | |
| V_{c}/F~CL/F | 89.12 | (10.41%) |
| V_{c}/F~Vₚ/F | 68.34 | (11.88%) |
| Vₚ/F~CL/F | 54.36 | (12.56%) |

RSE: relative standard error reported on the approximate standard deviation scale (SE/variance estimate)/2
CV: coefficient of variation
Kₐ: first-order absorption rate constant
Lag: absorption delay
V_{c}: central volume
Vₚ: peripheral volume
Q: intercompartmental clearance
F: bioavailability
CL: central clearance
D: duration of zero-order absorption

PK-RO Model Description

The PK model was linked to the RO model to generate the PK-RO model. The RO equation used (as described above) was the following:

RO=(Emax*Cp)/(Cp+EC₅₀)

Where RO=Receptor occupancy (%); Emax=96.16; Cp=Compound (I) concentration in central compartment of the model (ng/mL); EC₅₀=0.29 ng/mL.

### Example 4: RADIOLIGAND BINDING ASSAY

Human histamine H3 receptor membrane (PerkinElmer) were incubated with 1.0 nM [³H]—N-α-methylhistamine (PerkinElmer) in the presence or absence of increasing concentrations of ligands for H3 receptor competition binding. The binding incubations were in a final volume of 0.1 ml buffer (50 mMTris pH 7.5, 5 mM MgCl₂) at 28° C. for 120 minutes. Thioperamide (10 μM) was used to define non-specific binding.

All binding reactions were terminated by transferring 70 μl binding reaction from the reaction plate into filtration plates (Zeba 96-well Spin Desalting Plates, Thermo Scientific), followed by centrifugation at 1000 g for 2 min to collect the protein with bound radioligand. 200 μl of microscint-40 were added to determine the bound radiolabel by a Wallac Microbeta Trilux 2450 (Perkin Elmer). For all radioligand competition binding assays, IC₅₀ values and Hill slopes were determined by GraphPad Prism: log(inhibitor) vs. response with variable slope. Ki was calculated using the equation in Biochem. Pharmacol. 1973. 22 (23), 3099-3108, from Cheng and Prusoff: Ki=IC₅₀/{1+([radioligand]/Kd)}.

Results:

Compound (I), pitolisant and bavisant inhibited the binding of [³H]—N-α-methylhistamine at human recombinant H3R. The Ki values obtained from these binding experiments are 8.5, 153 and 102 nM for LML134, pitolisant and bavisant, respectively.

Conclusion: Compound (I) shows low nanomolar affinity at human H3 receptors. The affinity is 18 fold higher compared to pitolisant and 12 fold higher compared to bavisant.

### Example 5

5.1. Rat PK and PD (Tele-Methylhistamine)

Male Sprague-Dawley rats (Shanghai SLAC Laboratory Animal Co. LTD) of 6-7 weeks age were used. Animals were housed in a temperature and humidity-controlled environment on a 12 h light/dark. All animals had free access to food and water. All procedures were approved by Institutional Animal Care and Use Committee of ChemPartner Co., LTD (IACUC Protocol NO: A998HL0002).

For IV or PO experiments compounds were dissolved in citrate buffer (pH 3.5). IV bolus dose was administered via foot dorsal vein, and PO dosing via oral gavage.

For sampling, animals were restrained manually. Blood samples (approximately 150 μL/sample) were collected via tail vein at pre-dose and at post-dose for PO and IV arms. Blood samples were placed into tubes containing EDTA and centrifuged at 2000 g for 5 minutes at 4° C. to obtain plasma samples within 15 minutes of sampling. Following centrifugation, the resulting plasma was transferred to new tubes and snap frozen in dry ice and before transferred into −80° C. freezer pending bioanalysis.

Plasma concentrations of compounds were determined by a high performance liquid chromatography coupled with mass spectrometry (HPLC-MS/MS) method using an Agilent 6410, triple quadripole mass spectrometer (Mobile phase A: H₂O— 0.025% FA-1 mM NH₄OAC Mobile phase B: MeOH— 0.025% FA-1 mM NH₄OAC, column: Ultimate XB-C18 (2.1×50 mm, 5 μm with a flow rate of 0.45 mL/min and oven temperature of 40° C.) and dexamethasone as internal standard.

An aliquot of 30 μL plasma sample was mixed with 30 μL IS (Dexamethasone, 300 ng/mL) first, and later with 150 μL acetonitrile (ACN) for protein precipitation. Briefly, the mixture was vortexed for 2 minutes and then centrifuged at 12000 rpm for 5 minutes. A 5 μL supernatant was injected onto LC-MS/MS for analysis.

The PK parameters were determined with non-compartmental analysis by WinNonlin® Professional 6.2. The time points used to determine the terminal T½ were selected by the best fit model of WinNonlin. Manual selection of time points was applied when the best fit model was deemed not optimal by visual inspection.

For bioanalysis of brain tissue tele-methylhistamine samples were further processed.

One aliquot of the stock solution was diluted using ethanol/phosphate buffer pH 7.4 (85:15, v/v) to achieve a series working solutions. The brain homogenate was centrifuged at 12000 rpm for 5 min at 4° C. Then 10 μL supernatant of the brain homogenate sample was added to 60 μL internal standard (d3-1-methyl-histamine, 20 ng/mL). The mixture was vortexed for 2 min and centrifuged at 12000 rpm for 5 min at 4° C. Finally, 60 μL supernatant was added and 3 μL supernatant was injected into LC-MS/MS for analysis (Agilent 6410, triple quadrupole mass spectrometer, Mobile phase A: H₂O— 0.1% NH₃, Mobile phase B: methanol— 0.1% NH₃, Column: Ultimate XB-C18 (2.1×50 mm, 5 μm, Flow rate: 0.45 mL/min, temperature: 40° C.).

5.2. Ex Vivo Receptor Occupancy Assay

Male Sprague-Dawley rats (from Shanghai SLAC Laboratory Animal CO. LTD) at the age of 5-6 weeks were used. The animals were housed in a temperature and humidity-controlled environment on a 12 h light/dark cycle with food and water available ad libitum. All the procedures were approved by Institutional Animal Care and Use Committee of Chempartner Co., LTD (IACUC Protocol NO: A998HL0076).

Compounds were dissolved in 50 mM citrate buffer (pH 3.5), or saline. The compound was then diluted stepwise to the final concentration with vehicle. Animals were dosed by p.o. or i.v. in 1 ml/kg bodyweight.

At each time point, rats were decapitated immediately. Intact brain was removed, immersed and washed in pre-cooled saline. After drying with filter paper, frontal cortex was dissected with a double-blade with an about 30° angle starting at ⅓ of anterior rat brain (weight of most brain tissues around 70±10 mg). Brain tissue was transferred into a pre-weighed tube with homogenizer beads. The tubes were weighed again and the tissue net weight was recorded. Tissue sampling was done on ice with intervals of 1 min. Then, 50 mM HEPES (Gibco® by Life Technologies) buffer were added (equivalent to 3.75 μl/mg tissue weight) followed by homogenization (frequency 30/sec for 20 sec, TissueLyser, Quiagen).

The crude homogenate samples of rat frontal cortex were used to measure H3 receptor binding as described above with [³H]—N-α-methylhistamine as radioligand. The protein concentration of each sample was determined by Pierce BCA Protein Assay Kit (Thermo).

The cortical homogenate was added to a 96-well plate containing [³H]—N-α-methylhistamine (0.1 nM), in a volume of 0.2 ml (final protein concentration 4 μg/μl), incubated at 28° C. for 15 min and the reaction was stopped by rapid filtration using a filtration plate (Millipore multiscreen GF/B plate). The filters were washed three times with ice-cold wash buffer (50 mM Tris, pH 7.5) and 250 μl of Microscint-40 were added followed by determination of the bound radiolabel with a Wallac Microbeta Trilux 2450 (Perkin Elmer). Nonspecific binding was determined in the presence of 10 μM thioperamide. Each data point was obtained from a total of at least four animals. The inhibition of specific [³H]NAMH binding, calculated as relative to vehicle-treated samples, was determined to provide an indication of receptor occupancy by the compound. The dose-dependent % RO and time course were analyzed using GraphPad Prism. The dose response was fit using GraphPad Prism: log(inhibitor) vs. response with variable slope.

### Results:

### Rat PK

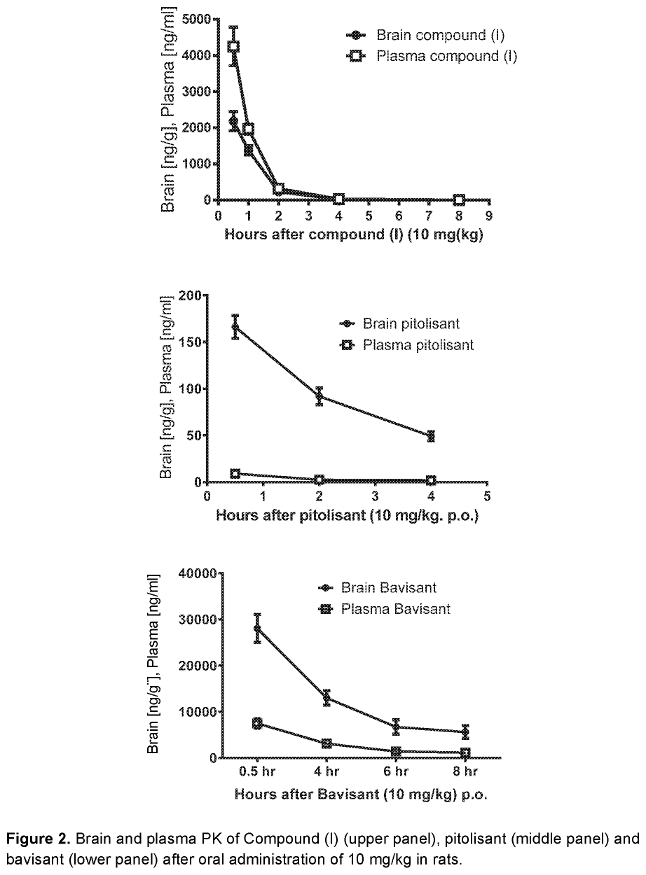

After administration of 10 mg/kg of the different compounds, total brain exposure was very high for bavisant, intermediate for Compound (I) and very low for pitolisant (FIG. 2). For Compound (I) brain exposure dropped at the 4 h time point to 21 ng/g and was close to detection limit at 8 h. In contrast the rate of exposure-decline for pitolisant and bavisant was much slower and high brain bavisant exposures could be detected at the 8 h time point (FIG. 2). These results implicate that the rapid brain penetration of bavisant and Compound (I) will entail an immediate central effect right after single compound administration. For pitolisant, repetitive cycles of compound administrations might be needed until sufficient levels are reached in the brain to inhibit H3 receptors. Further, the rapid drop in brain exposure for Compound (I) in contrast to bavisant, will not lead to RO at later time points and thereby will cause less insomnia in a clinical setting.

### Ex Vivo Receptor Occupancy Studies in Rats

In pre-experiments it was determined the dose for each compound that resulted in H3 receptor occupancy of 80-90% after oral dosing at 1 h after compound administration. The appropriate dose was found to be 10 mg/kg for LML134 and bavisant and 300 mg/kg for pitolisant.

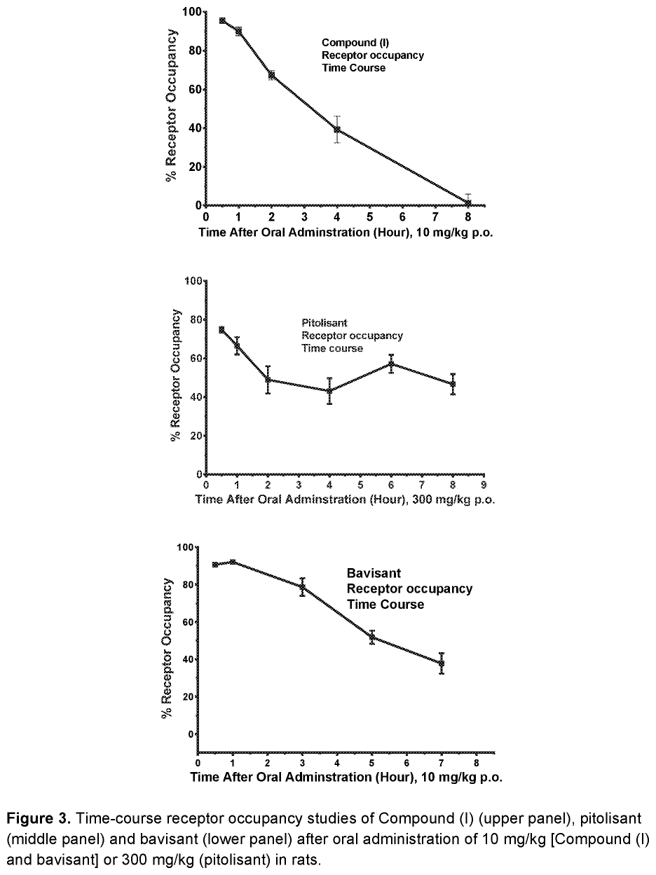

Using this data, a receptor occupancy (RO) time course study was run for all three compounds (FIG. 3).

Initial RO for LML134 was 95% at the 1 h time point and dropped to 39% at 4h. At 8 h, almost not RO was detectable.

Despite the high dose of 300 mg/kg for pitolisant the initial RO at 0.5 h hardly reached 90% and after a slight reduction of RO at t the 2 h time point, there was still considerable (40%) RO observed 8 h after compound administration.

Initial RO was high for bavisant (90%), but the decline in RO was slow and at 7 h still 37% RO was detected.

Conclusion:

Compound (I) and bavisant showed a high initial RO in contrast to pitolisant. Of all three compounds tested, only Compound (I) displayed a rapid decline of RO at 8 h indicating a fast disengagement of Compound (I) from the H3R. In the clinical setting Compound (I) will be administered in the morning and, it is expected that the lack of H3 inhibition 8-10 hours later would reduce the arousal effects implicated (and during the day wanted) with this mechanism. As a consequence, there will be less of an impact on sleep onset and, occurrence of insomnia will be minimized.

Ex Vivo PD Determination

The aim of these experiments was the assessment of an H3 RO dependent pharmacodynamic readout which is the inactive metabolite of histamine, tele-methylhistamine (tMeHA). The dose of each compound was chosen according to the results obtained in the PK experiments, which had shown that oral administration of 10 mg/kg Compound (I), pitolisant as well as bavisant induced significant brain exposures.

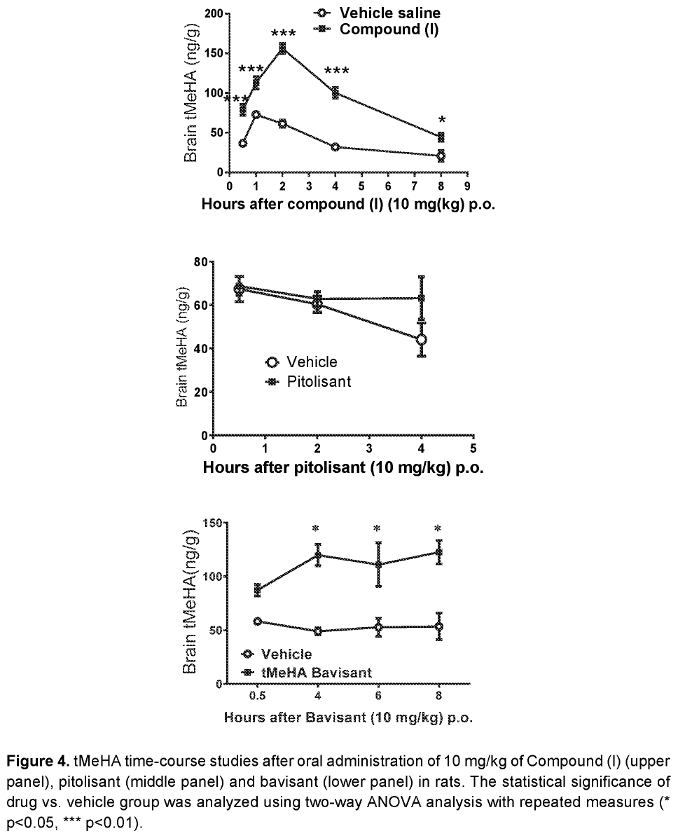

After oral administration of 10 mg/kg Compound (I) (FIG. 4, top panel) tMeHA levels are increased starting at 30 min, peaked at 2 h and stayed slightly elevated at 8 h. The longer lasting elevation of tMeHA compared to the rapid decline of RO (FIG. 3, top panel) might be caused by the longer half-life of tMeHA compared to histamine. After release, histamine has a half-life of about 20 min. Histamine is turned into the inactive metabolite tMeHA by the enzyme histamine-N-methyltransferase. tMeHA in the brain undergoes oxidative deamination through a monoamine oxidase (MAO-B) and an aldehyde dehydrogenase and finally t-methyl-imidazoleacetic acid is formed, a process that has a half-life of about 2-3 hours. Deconvolution of the PD data suggests that histamine is released, for about 2 h, after compound administration but due to the slower metabolism of the measured tMeHA, this PD marker stays elevated for up to 8 h.

After oral administration of 10 mg/kg pitolisant no increase of tMeHA above baseline level were observed. At the 4 h time point there was a trend for the pitolisant treatment group that tMeHA stayed at the same level as at the earlier time points, while tMeHA in the vehicle group decreased.

10 mg/kg bavisant induced a delayed increase of tMeHA that was only observed at 1 h after administration and stayed elevated over the entire observation period of 8 h without any sign of decline.

Conclusion

In contrast to the long lasting PK, RO and PD profile of bavisant and pitolisant, a very different profile is observed for Compound (I). Compound (I) has a short lasting PK and excellent brain penetration, that is observed immediately after compound administration. This PK is well reflected in a RO time course that starts with high receptor occupancy, that is needed for efficacy, going back, in rats, to 50% at 4 h and 0% at 8 h. As a consequence of the receptor occupancy, histamine is released and then turned into its inactive metabolite tele-methylhistamine. In line with the short PK and RO, these PD effects are also very transient. In view of the PK, RO and PD profile of Compound (I), this compound is expected to clinically translate into rapid-onset highly active arousal effects, without causing insomnia at bed time, since the efficacy of the compound is short lasting. In summary, less insomnia, and also less other side effects such as vivid dreams caused by long lasting RO, are thus expected for Compound (I) in contrast to bavisant and pitolisant. The data presented for pitolisant suggest that it will take repetitive administrations to establish central levels high enough to cause H3 inhibition and this levels will stay high almost unchanged also during night time.

### Example 6: Clinical Study

Study

A randomized, double-blind, placebo controlled, multi-center, seamless, combined proof of concept and dose-ranging study to assess safety, pharmacokinetics and efficacy of Compound (I) in Parkinson's disease patients with excessive daytime sleepiness.

Study Purpose

The main purposes of this study are to evaluate the safety, efficacy and to characterize the dose-response relationship of Compound (I) on patient reported sleepiness in Parkinson's disease (PD) patients measured by the Epworth Sleepiness Scale (ESS).

Study Objectives and Endpoints All study objectives will be evaluated based on combined results of Part A and Part B

| Primary objective(s) | Endpoints related to primary objective(s) |
|---|---|
| To characterize the dose-response | Change from baseline to Day 28 in ESS |
| relationship of Compound (I) to reduce | score |
| patient reported sleepiness | |
| Secondary objective(s) | Endpoints related to secondary objective(s) |
| To evaluate the safety and tolerability of | Treatment emergent adverse events and |
| Compound (I) | their corresponding severity, adverse |
| | events leading to discontinuation, and |
| | serious adverse events |
| To characterize the percentage of | ESS clinical response defined as ESS |
| patients with a clinical response on | absolute decrease from baseline of at least |
| patient reported sleepiness to | 3.0 points at Day 14 and 28 |
| Compound (I) compared to placebo | |
| Exploratory objective(s) | Endpoints related to exploratory objective(s) |
| To compare the effect of Compound (I) | Change from baseline to Day 28 in ESS |
| versus placebo on patient reported | and ISCS |
| sleepiness in levodopa and dopamine | |
| agonist users | |
| To assess the effect of Compound (I) | Significant other ESS at Baseline and on |
| compared to placebo on caregiver | Day 28 |
| evaluation of sleepiness | |
| To explore the effect of Compound (I) | Clinical Global Impression-Improvement |
| compared to placebo on global clinical | (CGI-I) scale score on Day 28 |
| impression of sleepiness | |
| To explore the effect of Compound (I) | Cognition/Attention tests (e.g. reaction time |
| compared to placebo on psychomotor | test, sustained attention task) at baseline, |
| function, attention, sustained attention, | on Day 14 and Day 28 |
| and other cognitive functions | |
| To assess the effect of Compound (I) | Number and duration of daytime naps and |
| compared to placebo on daytime naps | unintentional sleep episodes recorded daily |
| (including intentional and unintentional | from Baseline to Day 28 in the sleep diary. |
| naps) | Number and duration of daytime naps and |
| | unintentional sleep episodes measured by |
| | a wearable activity monitor from Day 1 to |
| | Day 28 |

Study Design

Overall Study Design:

This is a seamless, combined, non-confirmatory Ph2a or proof of concept (PoC) and Ph2b or dose range finding (DRF) study in PD patients with excessive daytime sleepiness (EDS). The study has two parts. Part A is the PoC study aiming to provide early evidence of safety and efficacy of Compound (I) in PD patients with EDS. Part B will provide additional data to establish the dose-response relationship of Compound (I) in the same patient population. Once the planned number of patients have completed Part A, an interim analysis will be performed. Recruitment will not be halted during the interim analysis, i.e. once a sufficient number of patients are recruited into Part A, recruitment will continue into Part B of the study. If Compound (I) demonstrates no prohibitive safety or tolerability signals, and clinically meaningful efficacy in Part A based on results of the interim analysis, Part B of the study will continue. Results of the entire study (Part A+Part B) will be used in combination to estimate doses for Ph3 studies using MCP-modeling.

An unblinded external Data Monitoring Committee (DMC) will review safety, tolerability and efficacy data every 6 months and at the interim analysis.

The study will use a randomized, double-blind, placebo controlled, parallel group, multi-center design. All study procedures will be identical in both study parts, therefore only one assessment schedule will be utilized.

Approximately 301 patients will be enrolled into the study: 112 patients into Part A and an additional 189 patients into Part B.

In Part A, patients will be randomized into one of three treatment arms in a 2:1:1 ratio:
Placebo
Compound (I), D3 [10 mg] once a day in the morning
Compound (I), D5 [20 mg] once a day in the morning

In Part B, patients will be randomized into one of six treatment arms in a 1:2:2:1:2:1 ratio:
Placebo
Compound (I), D1 [2 mg] once a day in the morning
Compound (I), D2 [5 mg] once a day in the morning
Compound (I), D3 [10 mg] once a day in the morning
Compound (I), D4 [15 mg] once a day in the morning
Compound (I), D5 [20 mg] once a day in the morning

Study Periods and Related Procedures:

An initial, up to 28-day long screening period will include screening for eligibility including assessment of severity of sleepiness [ESS and Clinical Global Impression-Severity (CGI-S)], PD stage and general health by examining vitals, ECG, labs, and physical examination as listed in the assessment schedule. For a subset of patients, a practice session of the attention and cognitive tests will also be performed during one of the screening visits.

Eligible patients will be requested to return to the site for a baseline visit on Day-1. During the baseline visit, all safety and efficacy assessments will be completed as specified in the assessment schedule, including the ESS. At selected sites, a subset of patients (approximately 50% of all patients, distributed proportionally to both study parts) will also participate in additional assessments aiming to provide measures of their alertness and sleep by neuropsychological testing and actigraphy, respectively. This subset of patients will henceforth be referred to as the actigraphy subset. Patients in the actigraphy subset will complete the attention and cognition tests during the baseline visit, while the patient is in the ‘ON’ state, in addition to all other safety and efficacy assessments specified in the assessment schedule. Once all baseline assessments are completed, patients can leave the site and return the next morning for dosing.

Patients will receive the first dose of study medication on Day 1 in the morning at around 09:00. Once all safety and PK assessments (PK sampling predose only) are completed and judged satisfactory by the investigator, patients may leave the site.

During the outpatient period (Day 2-Day 27) patients will take the study medication at home in the morning shortly after waking up, except for on Day 14, when patients will take the study drug at the clinic in the morning.

Safety will be evaluated on Day 7 and Day 14 along with the PK on Day 14 (PK sampling at predose and at 2 and 4 hours post dose) as specified in the assessment schedule. In addition, efficacy assessments (ESS in all patients and also cognition and attention tests in the actigraphysubset) will also be performed on Day 14. During the outpatient period, patients will also be requested to complete a sleep diary daily. Patients in the actigraphy subset will also be requested to wear a portable activity monitor (actigraph) to measure the number and duration of planned daytime naps and unintentional sleep episodes and evaluate their night-time sleep.

Patients will return to the site on Day 28 for final safety and efficacy assessments along with PK sampling (predose and at 2 and 4 hours post dose) as specified in the assessment schedule. Study drug will be taken at the clinic in the morning on Day 28. The ESS will be administered at the same time of the day as during the baseline visit. Patients in the actigraphy subset will also undergo cognition and attention tests performed, while the patient is in the ‘ON’ state. the same way and at the same time of the day as during Baseline. The Investigator must exercise reasonable effort to perform these tests at the same time versus anti-parkinsonian medication as during Baseline. Safety assessments will be performed as specified in the assessments schedule. If all safety assessments are completed with satisfactory results, patients may leave the site. Patients will return to the site 7-14 days after taking the last dose of study drug for a safety follow-up visit (Day 35-42). If all safety assessments are completed with satisfactory results, patients will be discharged from the study.

If the patient has an assistant, the patient assistant will be required to complete the Significant Other ESS at Baseline, Day 14 and Day 28. The scale might be completed at the clinical site during a face to face visit or over the phone. However, for each patient assistant the scale has to be completed in the same way throughout the study. The patient assistant is a person, who is willing to participate in the study and is able to understand and comply with study procedures in the local language and able to provide informed consent for themselves. The patient assistant does not need to be a primary or formal caregiver and can include a family member or other individual who helps with the patient's activities of daily living. The patient assistant does not need to live in the same household as the patient, but needs to spend sufficient time with the patient (minimum 2 days per week) to allow reliable assessment of the patient's sleepiness.

Patients are not required to spend the night at the site before or after the other inpatient visits, if they live close to the clinical site. However, for all sites, if patients live far from the clinical site, an overnight stay will be provided at the site or a hotel nearby for the patient and the patient's assistant. It is important to avoid a long commute in the morning and the consequential short overnight sleep before assessment days.

Population

The study population will be comprised of PD patients with excessive daytime sleepiness. Patients who participated in Part A of the study are not eligible to participate in Part B.

Inclusion criteria
1. Signed informed consent must be obtained prior to participation in the study.
2. Male and female patients over 30 years of age
3. Patients with clinically established Parkinson's disease.
4. Anti-parkinsonian medication (total daily dose and dosing regimen) has to be stable for at least four weeks prior to baseline and no changes should be foreseen for the duration of the study.
5. Medical history (at least 3 months) of excessive daytime sleepiness and an ESS score 13 at screening.
6. Outpatients, residing in the community (nursing home patients are not allowed).
7. Able to communicate well with the investigator, to understand and comply with the requirements of the study.
8. If the patient is using any psychotropic medication that may impact daytime sleepiness the daily dose and regimen of such medication has to be stable for at least 4 weeks for sedatives, hypnotics and anxiolytics and for at least 8 weeks for anti-depressants and anti-psychotics prior to Baseline and no changes should be foreseen for the duration of the study. Patients, who use quetiapine might be enrolled, if they take quetiapine only in the evening.

### Statistical Model, Hypothesis, and Method of Analysis

This study will be conducted in two parts. Part A will contain a futility assessment to assess the efficacy of Compound (I) 10 mg and 20 mg doses compared to placebo.

### Analysis of Part A:

The primary objective is to assess the performance of the ESS at the end of the treatment period, i.e. study day 28. An interim analysis will be performed with 112 patients (Placebo: Compound (I) 10 mg: Compound (I) 20 mg=56:28:28). A mixed effect model for repeated measures (MMRM; e.g. in Mallinckrodt C H, Clark W S, David S R (2001) Accounting for dropout bias using mixed-effects models. Journal of Biopharmaceutical Statistics; 11:9-21) will be performed over ESS data. This MMRM model will contain treatment group, baseline ESS score, visit, availability of sleep lab in treated site, levodopa/dopamine agonist use and treatment group×visit as covariates, with an unstructured covariance matrix.

Futility analysis: The study will be stopped due to futility if the improvement in change from baseline ESS score is less than 1 unit. In order to evaluate futility, the pooled treatment effect from Compound (I) 10 mg and Compound (I) 20 mg will be compared versus placebo within the abovementioned MMRM model. The least square mean difference of pooled treatment versus placebo will be evaluated to test the futility.

Analysis of Part B: The null hypothesis of a flat dose-response curve for the change from baseline in ESS score will be tested at a significance level of 5% against the one-sided alternative hypothesis of a non-constant dose response curve using the MCP-Mod methodology (e.g. in EMA's Qualification Opinion on MCP-Mod dated Jan. 23, 2014: EMA/CHMP/SAWP/757052/2013).

Hence, the following null and alternative hypotheses will be tested:
H₀₁: there is no dose-response relationship for Compound (I) (i.e. the dose response relationship is flat).
H₁₁: there is a dose-response relationship for Compound (I) (i.e. the dose response relationship is not flat).

Generalized MCP-Mod (Pinheiro et al, Stat in Med, 2014, 33(10), 1646-1661) will be applied based on the output from a mixed effect model for repeated measurements (MMRM). The MMRM model will contain treatment group, baseline ESS score, visit, availability of sleep lab in treated site, levodopa/dopamine agonist use, and treatment groupxvisit as covariates, with unstructured covariance matrix. To perform generalized MCP-Mod the least square mean values at each individual dose for Day 28 and associated covariance matrix will be obtained from the MMRM.

For each candidate model a contrast test statistic, based on a linear combination of the treatment estimates per dose will be derived. The contrast coefficients will be chosen to maximize the power to detect pre-specified candidate models. For this purpose, Emax, sigmoid Eₘₐₓ, linear and beta dose-response shapes will be selected. For the Emax model, ED₅₀=2.5 will be used, for the sigmoid Emax models three shapes with (ED₅₀, h)=(5, 5),(7.5, 5) and (13, 10) will be used and for the β-model=(2, 1) will be used with the scale parameter=24.

The Formulae for the models are as follows:

TABLE 1
Candidate dose-response models for Dose selection hypotheses

| Candidate | | |
|---|---|---|
| model | Formula η(d) | Parameter values |
| Emax | E₀ + Emax × d/(d + ED₅₀) | ED₅₀ = 2.5 |
| | | (ED₅₀, h) = (5, 5) |
| Sigmoid Emax: | E₀ + Emax × d^{h}/(d^{h} + | (ED₅₀, h) = (7.5, 5) |
| | ED₅₀^{h}) | (ED₅₀, h) = (13, 10) |
| Linear: | E₀ + δd | |
| Beta: | E₀ + Emax × B(δ₁, δ₂)x^{δ}₁ | (δ₁, δ₂) = (2, 1) with scale |
| | (1 − x)^{δ}₂ | between 0 and 24 |

E₀ = the expected placebo effect; Emax = the maximum change in effect over placebo; ED₅₀ = the dose at which 50% of Emax is achieved; h = the hill parameter; B(δ₁, δ₂) = (δ₁ + δ₂)^{δ}₁^{+δ}₂/δ₁^{δ}₁δ₂^{δ}₂; x = d/scale.

The same shapes will be used to derive the contrasts. All contrasts will be combined into one contrast matrix. The global test decision is based on the maximum of all contrast test statistics. A critical value q controlling the type I error rate can be derived from the fact that the contrast test statistics approximately follow a multivariate t distribution. If the maximum contrast test statistic exceeds the critical value q, the overall null hypothesis of a constant dose-response curve is rejected and further estimation steps can be followed to determine the dose-response curve and the doses that will achieve the target clinical effect.

Bootstrapping will be used to estimate the dose-response curve and to derive confidence intervals. Bootstrap simulation will be performed using the multivariate normal distribution of the obtained estimates for the different doses and estimated covariance matrix from the MMRM analysis. Generalized least squares fitting of the resulting simulated values will be utilized (Pinheiro et al, Stat in Med, 2014, 33(10), 1646-1661).

Sample Size Calculation

Sample Size Justification for Futility Assessment:

Futility rule of less than 1 unit improvement in change from baseline ESS score compared to placebo is justifiable for the following reasons:

A. From the available literature and published articles an improvement of 1 unit in ESS is assumed to be minimal evidence of efficacy against placebo.
B. With the given sample size (i.e. 112) any chance of missing a true effect size of 2.5 units or above is less than 10%. The following table is computed from operating characteristics of the futility rule with an SD of ESS score of 6.

TABLE 2
Operating Characteristics: Futility Rule

| Effect Size | Chance of hitting futility (%) |
|---|---|
| 2.5 | 9.3 |
| 2.6 | 7.9 |
| 2.7 | 6.7 |
| 2.8 | 5.6 |
| 2.9 | 4.7 |
| 3.0 | 3.9 |

Sample Size Justification for Dose Finding:

The primary objective of this study is to characterize the dose response relationship among Compound (I) doses (2, 5, 10, 15 and 20 mg) and placebo for the primary efficacy parameter ESS measure at 28 days after randomization. The sample size for the primary analysis was determined with the software ADDPLAN DF, version 4.0, with settings for average power function and model based contrasts.

The sample size is derived to detect a dose-response of at least 95% power and a one-sided alpha of 5%. It is assumed that there will be no change in ESS in the Placebo arm, while a maximum treatment effect for Compound (I) is expected to be 3 points higher than placebo after 28 days of treatment.

Due to the two stage design, the final (unbalanced) allocation ratio of 11:6:6:7:6:7 is expected, corresponding to treatment arms placebo: 2 mg: 5 mg: 10 mg: 15 mg: 20 mg. The initial randomization of patients in Part A will be 56:28:28 corresponding to treatment arms placebo: 10 mg: 20 mg. Subsequently, the initial doses randomized to Part A will be slightly higher in proportion to the other doses from Part B.

Under these assumptions, a sample size of 301 subjects in total will be required, randomized to 77:42:42:49:42:49 subjects corresponding to placebo: 2 mg: 5 mg: 10 mg: 15 mg: 20 mg treatment arms.

TABLE 3
Sensitivity of power to changes in assumptions for N = 301

| True treatment difference for best dose of | SD | Average | Minimum |
|---|---|---|---|
| Compound (I) vs Placebo (mg) | (mg) | power | power |
| 3 | 5 | 99% | 98% |
| 3 | 6 | 95% | 92% |
| 2.5 | 5 | 95% | 92% |
| 2.5 | 6 | 87% | 82% |

Calculations were performed using the ADDPLAN-DF version 4.0.

Power calculations do not take into account the futility interim analysis, which will have a minimal effect on the overall power.

1-14. (canceled)

15. A method for treating excessive daytime sleepiness associated with Parkinson's disease, in a subject, in need thereof, comprising administering to said subject an effective amount of 1-(1-methyl-6-oxo-1,6-dihydropyridazin-3-yl)piperidin-4-yl 4-cyclobutylpiperazine-1-carboxylate, or pharmaceutically acceptable salt thereof.

16. A method according claim 15, wherein said 1-(1-methyl-6-oxo-1,6-dihydropyridazin-3-yl)piperidin-4-yl 4-cyclobutylpiperazine-1-carboxylate, or pharmaceutically acceptable salt thereof, is administered in the form of a pharmaceutical composition further comprising at least one pharmaceutically acceptable excipient.

17. A method according to claim 15, wherein said 1-(1-methyl-6-oxo-1,6-dihydropyridazin-3-yl)piperidin-4-yl 4-cyclobutylpiperazine-1-carboxylate, or pharmaceutically acceptable salt thereof, is administered in combination with one or more further pharmaceutical active ingredient.

18. A method according to claim 17, wherein the one or more further pharmaceutical active ingredient is a wakefulness-promoting agent.

19. A method according to claim 17, wherein the one or more further pharmaceutical active ingredient is selected from the group consisting of levodopa; the combination of levodopa and pergolide; the combination of levodopa and cabergoline; the combination of levodopa and ropinirole; the combination of levodopa and carbidopa; the combination of levodopa and entacapone; the combination of levodopa and benserazide; and the combination of levodopa and pramipexole; or pharmaceutically acceptable salts thereof.

20. A method according to claim 15, wherein the method is combined with psychological therapy or behavioral therapy.

21. A method according to claim 20, wherein said behavioral therapy is cognitive behavioral therapy focused on sleep hygiene rules.

22. A method according to claim 20, wherein said behavioral therapy is computer-assisted.

23. A method according to claim 15, wherein excessive daytime sleepiness coexists with one or more sleep disorders associated with Parkinson's disease.

24. A method according to claim 15, wherein said 1-(1-methyl-6-oxo-1,6-dihydropyridazin-3-yl)piperidin-4-yl 4-cyclobutylpiperazine-1-carboxylate, or pharmaceutically acceptable salt thereof, is administered in an amount of from 0.1 mg/day to 50 mg/day.

25. The method of claim 24 wherein said 1-(1-methyl-6-oxo-1,6-dihydropyridazin-3-yl)piperidin-4-yl 4-cyclobutylpiperazine-1-carboxylate, or pharmaceutically acceptable salt thereof, is administered in an amount of from 1 mg/day to 20 mg/day.

26. A method according to claim 15, wherein said 1-(1-methyl-6-oxo-1,6-dihydropyridazin-3-yl)piperidin-4-yl 4-cyclobutylpiperazine-1-carboxylate, or pharmaceutically acceptable salt thereof, is administered orally.

27. A method according to claim 15, wherein said Parkinson's disease coexists with a psychiatric disorder.

28. A method according to claim 15, wherein said Parkinson's disease is early-stage of Parkinson's disease, mid-stage Parkinson's or advanced-stage of Parkinson's disease.
